(19) United States
(12) Patent Application Publication
(10) Pub. No.: US 2020/0378951 A1
(43) Pub. Date: Dec. 3, 2020

(54) IMPEDANCE ASSAY

(71) Applicant: Cell Therapy Catapult Limited, London (GB)

(72) Inventors: Damian Marshall, London (GB); Alexander Chan, London (GB); Adeyemi Afuwape, Surrey (GB)

(21) Appl. No.: 16/463,931

(22) PCT Filed: Nov. 23, 2017
(86) PCT No.: PCT/EP2017/080202

§ 371 Date: May 24, 2019

(30) Foreign Application Priority Data
Nov. 24, 2016 (GB) 1619909.3

(51) Int. Cl. G01N 33/50; G01N 33/543

(52) U.S. Cl. CPC G01N 33/5011; G01N 2500/10; G01N 33/5438

(57) ABSTRACT

The present invention provides a method of determining the cytotoxic effect of an effector molecule or cell on a target cell presenting a heterologous peptide comprising: (a) incubating said effector molecule or cell with said target cell, wherein said target cell is immobilised on a substrate comprising at least one pair of electrodes; and (b) determining the impedance during and/or after step a) wherein a decrease in impedance is indicative of target cell death. The invention further provides the use of impedance to determine the cytotoxic effect of an effector cell and/or molecule on a target cell presenting a heterologous peptide and a kit for use in the method of the invention, comprising (a) a target cell presenting a heterologous peptide or a target cell and a heterologous peptide or a nucleic acid comprising a nucleotide sequence encoding said heterologous peptide; (b) a capture molecule for immobilising said target cell to a substrate; (c) instructions for use of (a) and (b) in a method of the invention; and optionally (d) a substrate comprising at least one pair of electrodes; and/or (e) an effector molecule and/or cell which specifically binds to the heterologous peptide presented by the target cell, or an effector cell and a nucleic acid comprising a nucleotide sequence encoding a receptor which specifically binds to the heterologous peptide presented by the target cell.

The present invention relates generally to methods of determining and/or measuring the cytotoxic effect of an effector cell or molecule on a target cell by determining and/or measuring impedance. More particularly, the invention relates to methods of determining the cytotoxic effect of an effector cell or molecule on a target cell presenting a heterologous peptide, particularly a heterologous tumour associated antigen by measuring and/or determining impedance. The invention further relates to the use of impedance to determine the cytotoxic effect of an effector cell or molecule on a target cell, particularly a target cell presenting a heterologous peptide (e.g. a tumour associated antigen).

Immunotherapy, which encompasses the specific targeting of cancer cells by immune cells, is rapidly emerging as a plausible and promising treatment approach for cancer. Particularly, the use of adoptive T-cell therapies for the treatment of both haematological cancers and solid tumours is one of the fastest growing areas in the cell and gene therapy field, where the genetic modification of T cells to determine antigen binding specificity or to enhance function is often carried out.

T cells may be modified to express exogenous receptors, e.g. T-cell receptors (TCRs) or Chimeric Antigen Receptors (CARs), which may confer a particular antigen binding specificity upon the modified T cell, to direct binding of the modified T cell to a cancer cell. In principle, any cell surface molecule can be targeted by using a CAR immunotherapy, thus overriding tolerance to self-antigens and providing a treatment which is not reliant on the MHC status of a patient. Using T cells engineered to express a chimeric antigen receptor targeting CD19, recent trials have demonstrated remarkable clinical responses in leukaemia and lymphoma patients. In contrast, T-cells modified to express a TCR can target intracellular proteins through the presentation of their fragments on the cell surface by HLA molecules. The chosen approach for an immunotherapy thus is generally dependent upon whether the target antigen is expressed on the cell surface or is expressed intracellularly and presented on the cell surface in the form of an HLA complex.

It will be appreciated that the development of in vitro assays to determine the potency of any potential immunotherapies is essential. Firstly, it is important to assess immunotherapies during development and to demonstrate an initial in vitro cytotoxic effect against a target cell of interest (e.g. a cell expressing a target antigen or antigen/HLA complex) before the therapy proceeds to testing in animal models or to clinical trials. Further, given the potential variability between different product batches, e.g. due to differences in transduction efficiency and TCR/CAR expression levels, it is important that manufactured product is subjected to in vitro testing before in vivo use. The use of a patient's own cells may result in the incorporation of further possible variability in the efficacy of an immunotherapy product, which makes in vitro testing of the product prior to implantation a necessity. Given the wide range of immunotherapies which are under development, and which target numerous different antigens, it is desirable that a generic in vitro test be developed which can effectively and efficiently determine or measure the efficacy (e.g. the cytotoxic effect) of a modified immune cell (e.g. T-cell) product upon a target cell of interest.

Traditional assays which measure the cytotoxic effect of an immune cell upon a target cell of interest, usually employ one of two methods.

The first method involves measuring the activation of effector cells (i.e. the immune cells) or their secretion of cytotoxic molecules (e.g. perforin, granzymes etc). Particularly, assays measuring TNFα or IFNγ levels have been used to determine effector cell activation. Whilst such assays are useful for the biochemical characterisation of effector cells, their correlation with target cell killing may not be direct or accurate.

The second method involves monitoring the target cells, rather than the effector cells and particularly concerns measuring the release of substances from the target cells upon their lysis. Such methods usually involve the measurement of a pre-loaded label, such as Cr⁵¹ or a fluorescent label, or the measurement of an endogenous biomolecule such as GAPDH, LDH etc. These assays may be problematic since there may be artefacts associated with labelling and the time frame for carrying out the experiment is restricted in view of the constant leakage of labels from the cells.

Hence, the traditional assays for monitoring the cytotoxic effects of immune cells are generally at best semi-quantitative, and often require complex pre-labelling of cells or are based on the detection of surrogate markers. Further, these assays only provide end-point data and there is no ability to obtain any real time measurements of cytotoxic effect.

Electronic methods for analysing cellular compositions have been in use for many years. Particularly, impedance based assays have been utilised for a variety of different applications, including measuring cell migration, counting cells or measuring cell growth, as in U.S. Pat. No. 7,470,533 (incorporated herein by reference). More recently, impedance based assays have been developed to investigate the cytotoxic effect of immune cells upon target cells. These assays are based upon the principle of cellular impedance, where adherent cells act as insulators impeding the flow of an alternating microampere electric current between electrodes. A change in measured impedance within such a system (i.e. due to a change in the current flow) between and amongst electrodes can identify a change in the quantity or presence of cells. Thus, target cells which adhere to a substrate comprising electrodes may impede the flow of the current and the presence of adherent target cells on the substrate hence results in increased impedance due to their insulating effect. The addition of immune effector cells which specifically bind to the adherent target cells, resulting in target cell death, decreases the impedance since fewer target cells are present to produce an insulating effect. Thus, impedance can be used to measure target cell death and the number of target cells present on the substrate. The method is sensitive and can also allow the measurement of real time target cell killing.

Cerignoli et al (The FASEB Journal, vol 30, No. 1 Supplement, 715.4, 2016), used impedance to determine the cytotoxic effect of Primary Blood Mononuclear Cells (PBMCs) mediated by an EpCAM/CD3 bispecific T cell engager (BiTE) antibody on several carcinoma cell lines with different expression levels of the EpCAM membrane protein. Further, the cytotoxic effect of a T-lymphoblast cell line on immobilised lymphoma cells was investigated using impedance. However, the impedance assays currently available mainly utilise adherent cancer cells, non-adherent malignant B cells or transformed cell lines expressing endogenous or viral antigens as target cells, to which the used effectors bind. Whilst these assays provide accurate information regarding the cytotoxic effect of effector cells which bind to expressed tumour associated antigens on the tested cancer cells, they do not currently provide a generic assay which is capable of testing the binding of an effector cell with any binding specificity to any target cell presenting its target peptide.

The present inventors have developed an alternative improved impedance based assay, which is capable of generic use to determine the cytotoxic effect of any effector (e.g. cell) having a binding specificity for any antigen (i.e. not just those which may be endogenously expressed on the cancer or virally transformed cell lines tested). Particularly, the inventors have utilised target cells in the assay (and more particularly non-adherent target cells, e.g. antigen presenting cells) which present (e.g. display) a heterologous peptide or tumour associated antigen (e.g. non-viral) to which an effector cell specifically binds. In this way, it is possible to use the assay to test any modified effector cell product which binds to any tumour associated antigen (e.g. non-viral). The use of a heterologous peptide expressing or pulsed target cell and particularly, a non-adherent pulsed target cell, was not previously contemplated in the impedance assays of the art and the previously developed assays mainly investigated effector cells which specifically bound to cancer or malignant cells or virally transformed cells expressing endogenous tumour associated or viral antigens.

Further, the inventors have identified that the generic assay can be used to determine the effect of effector cells or molecules on target cells when different levels or amounts of heterologous peptide are displayed. This may allow the target cells to mimic expression levels of peptide which would be found in different disease conditions, at different disease stages or under different in vivo conditions. Particularly, the expression levels of an endogenous peptide (e.g. a tumour associated antigen in cancer cells or cell lines), may differ in vitro and in vivo, due to the very different conditions under which the cells are placed. Thus, utilising an adherent/non-adherent cancer cell or cancer cell line in an in vitro impedance assay and determining the effector cell or molecule effect may not necessarily reflect the effect which would occur in vivo due to the possible differing levels of expressed endogenous peptide targeted by the effector cell or molecule. The ability to tailor the amount of heterologous peptide displayed by the target cells in the presently developed generic assay addresses such problems and allows for a more accurate reflection of the cytotoxic effect which may occur in vivo. Thus, the amount of heterologous peptide displayed can be tailored to match that displayed on any naturally occurring cell under any condition. Accurate tailoring of the amount of peptide displayed/presented may be particularly achieved by pulsing heterologous peptide into a target cell (e.g. a non-adherent target cell).

Further, the ability to tailor target cells to display any amount of heterologous peptide, allows the assay to investigate any off target effects of the effector cell or molecule, by displaying low levels of peptide on the target cells. This may be particularly important where a tumour associated antigen is expressed at low levels on non-tumour cells.

Further, the inventors have identified that the use of target cells expressing a heterologous peptide, to which the effector cells specifically bind, results in a particularly efficient assay when the target cells are pulsed with a heterologous peptide prior to any attachment to a substrate. Thus, the inventors have shown that pulsing prior to target cell immobilisation results in a significantly improved cell index (i.e. target cell adherence to the necessary substrate) which allows a more accurate impedance measurement to be obtained during the assay, e.g. particularly for non-adherent target cells. This optimisation of the assay thus results in an unexpected and significant improvement of the modified assay.

As discussed briefly above, the cytotoxic impedance assays of the art primarily used adherent cell lines, primary cells or in one instance a non-adherent B cell line. The present inventors have developed a generic assay which can be particularly used with non-adherent target cells, presenting (e.g. displaying or expressing) any amount of heterologous peptide (as opposed to endogenous peptides e.g. endogenous tumour associated antigens or viral peptides in virally transformed cell lines), and particularly with any non-adherent antigen presenting cell, which as discussed below may be advantageously immobilised to a suitable substrate using an anti-CD40 antibody.

Hence, in a first embodiment, the present invention provides a method of determining the cytotoxic effect of an effector cell and/or molecule on a target cell (e.g. a non-adherent target cell) presenting a heterologous peptide comprising:
a) incubating said effector cell and/or molecule with said target cell, wherein said target cell is immobilised on a substrate comprising at least one pair of electrodes; and
b) determining the impedance during and/or after step a), wherein a decrease in impedance is indicative of a cytotoxic effect.

Thus, the method of the invention can be used to measure or determine whether an effector molecule or cell has any cytotoxic effect on a target cell presenting a heterologous or exogenous peptide (e.g. a tumour associated antigen), by measuring, detecting or determining impedance or a change in impedance. As indicated previously, the presence of target cells immobilised or bound to a substrate will increase the insulation between electrodes present within or on a substrate, which will reduce the flow of electrons between electrodes, and, which will in turn result in impedance. The addition of an effector cell or molecule which specifically binds to the heterologous peptide (e.g. tumour associated antigen) present on the target cell surface may result in a change in impedance, if the effector cell or molecule has a cytotoxic (killing) effect on the bound target cells. The effector cell or molecule preferably does not bind directly to the substrate or become directly immobilised to the substrate and hence the presence of the effector cell or molecule per se does not have an effect on impedance or has a minimal effect on impedance (e.g. has an impact on impedance of less than 5, 4, 3, 2 or 1%). It is only the binding of the effector to the target cell (where alternatively viewed, the effector is indirectly bound to the substrate through immobilised target cell interaction), that could affect impedance and only if that binding has a cytotoxic effect on the target cell. Thus, if the effector has a cytotoxic (i.e. killing effect) on the target cells, the number of target cells immobilised to the substrate will decrease, resulting in reduced impedance, as compared to the impedance detected before the addition of the effector molecule or cell. In this way, a reduced impedance is indicative of target cell death and thus of the cytotoxic effect of the added effector molecule or cell. If no change or no significant change is detected in impedance, or indeed if impedance increases, then it is likely that the effector molecule or cell may have little or no cytotoxic effect on the target cell (to which it may (or may not) specifically bind). An increase or no significant change (e.g. no significant decrease) in impedance indicates that significant target cell death has not occurred during or after the addition of the effector cell or molecule (and if an increase is impedance is detected, then target cell growth may have even occurred). This in turn is indicative of a lack of cytotoxic effect of the effector molecule or cell. Hence, the impedance detected is directly related to the number of cells immobilised to the substrate, which is in turn directly related to the cytotoxic effect of the effector cell or molecule.

As discussed previously, the “target cell” as referred to herein, may be any cell which is capable of presenting a heterologous peptide (e.g. a tumour associated antigen) on its cell surface. As discussed below, particularly, the target cell is non-adherent (e.g. a non-adherent antigen presenting cell). “Presenting” means displaying, having or comprising a heterologous peptide on the surface of the target cell, e.g. so that particularly at least a portion of the heterologous peptide is displayed or is present extracellularly. Thus, the heterologous peptide is “present” on the target cell surface (or at least partially present on the target cell surface). In this way, the heterologous peptide may be identified by any added effector cell or molecule which has specificity for the heterologous peptide as present on the target cell surface.

The “heterologous peptide” may be present on the target cell surface alone or comprised within or bound to another polypeptide, protein or complex (e.g. an endogenously expressed protein or complex, such as MHC/HLA).

It will be appreciated that particular peptides, e.g. particular tumour associated antigens, which are usually present within intracellular proteins, may only be present on the surface of a cell in combination with a particular MHC or HLA molecule (e.g. a MHC class I or II or HLA I or II molecule). For such peptides/antigens, it may be necessary to utilise an antigen presenting cell as the target cell (e.g. a non-adherent antigen presenting cell), where the antigen presenting cell is capable of presenting particular peptides (e.g. tumour associated antigens) together with MHC/HLA (class I or II) on the cell surface. Both professional and non-professional antigen presenting cells may be used as the target cell in the present invention. Particularly, the target cell may be a dendritic cell, a macrophage, a B cell or any other type of antigen presenting cell. Antigen presenting cell lines may be used in the present invention, e.g. to reduce assay variability arising from using a patient's own cells.

In one embodiment, the antigen presenting cell may be modified to reduce or prevent the formation of MHC/HLA complexes with endogenously expressed peptides, and thus to allow or increase the amount of MHC/HLA available for loading with heterologous or exogenous peptide (e.g. tumour associated antigen). Hence, target antigen presenting cells may be TAP deficient (or in another embodiment TAP competent). Thus, particularly, the peptide transporter associated with antigen processing (TAP) may be deficient or missing from an antigen presenting cell for use in the present invention. More particularly, T2 cells (formed from fusing T and B lymphoblasts) may be used in the present invention as target cells, e.g. T2 cells from ATCC, Cat #ATCC-CRL-1992. Alternatively, JY cells or C1R-A2 cells may be used. It will be appreciated by a skilled person that antigen presenting cells which are not modified to reduce or prevent formation of MHC/HLA complexes with endogenous peptides, may be treated during or prior to the method of the invention, to reduce the number of MHCs/HLA comprising or associated with endogenous peptides, e.g. by treating with acid, e.g. citric acid, more particularly citric acid monohydrate. It may further be desirable to incubate treated cells with beta-2-microglobulin. Protocols for such treatment are available (e.g. at BioProtocols, vol 6, 18, 2016). For other peptides, which may not require complex formation with MHC/HLA in order to be presented on the cell surface, it may be possible to use other cell types as the target cells, e.g. endothelial or epithelial cells. Any cell that can be genetically modified or pulsed to express a heterologous peptide on its surface can be used. Preferably, a target cell of the invention may be a non-adherent cell and particularly a non-adherent antigen presenting cell.

It will further be appreciated that the type of peptide and the type of target cell to be used may determine the effector which may be used in the assay (or vice versa). Thus, if a heterologous peptide is presented on the cell surface of an antigen presenting cell in a complex with MHC/HLA, it will be appreciated that an effector cell which may be used in the method may be a T cell expressing a TCR which specifically binds or potentially specifically binds to the heterologous peptide:MHC/HLA complex. This is discussed in detail further below.

A “peptide” in accordance with the present invention may comprise at least 3 amino acids, more particularly at least 5, 6, 7, 8, 9, 10, 11, 12, 13, 14, 15, 16, 17, 18, 19, 20, 21, 22, 23, 24 or 25 amino acids. Particularly, a peptide associated on the target cell surface with a MHC class I molecule may be between 7-10 amino acids in length and a peptide associated on the target cell surface with an MHC class II molecule may be from 13-21 amino acids in length. However, the terms “peptide”, “polypeptide” or “protein” are used interchangeably herein and mean a polymer of amino acids, not limited to any particular length. The term does not exclude modifications such as myristylation, sulfation, glycosylation, phosphorylation and addition or deletion of signal sequences. The terms “peptide”, “polypeptide” or “protein” thus mean one or more chains of amino acids, wherein each chain comprises amino acids covalently linked by peptide bonds.

The “heterologous” peptide as used in the method of the invention is a peptide which is not naturally expressed by the target cell, i.e. is exogenous. As discussed previously, the heterologous peptide is present on the surface of the target cell (e.g. at least partially present on the cell surface) and hence, the target cell as used in the invention presents a peptide which is not naturally occurring. The heterologous peptide is therefore not a tumour associated antigen which is endogenously expressed by any cancer cell or malignant cell when being used as the target cell. Further, heterologous peptide may not be a viral antigen which is endogenously expressed in a virally transformed cell line when being used as the target cell. Such TAAs or viral antigens may therefore be considered heterologous peptides only when pulsed or expressed in a target cell which does not typically express or present those TAAs or viral antigens. The heterologous peptide may be expressed by the target cell or may have been pulsed into the cell as a peptide. Thus, a nucleic acid molecule comprising a nucleotide sequence encoding the heterologous peptide may be transduced into the target cell or an artificially or recombinantly produced peptide may be used for target cell pulsing.

An expressed heterologous peptide may thus be encoded by an exogenous DNA or an RNA molecule. The introduced or exogenous nucleic acid molecule (e.g. DNA/RNA) may encode the peptide which is presented on the target cell surface, or alternatively may encode a peptide or polypeptide comprising the presented peptide, e.g. a longer peptide or polypeptide, which may be processed intracellularly, resulting in the presentation of the peptide. Thus, the presented peptide may be a fragment of the expressed peptide or polypeptide encoded by any introduced nucleic acid.

As discussed above, the peptide presented on the target cell may alternatively be introduced into the target cell by pulsing. The heterologous peptide for pulsing may be artificially synthesised or produced by recombinant expression in an appropriate host cell (e.g. a prokaryotic cell, such as E. coli, or an insect cell), by transforming the host cell, using a method as described below, with a nucleic acid molecule comprising a nucleotide sequence encoding the peptide. The artificial synthesis of peptides is well known in the art and any well-established method can be used to produce a peptide for pulsing into a target cell of the invention. Briefly, peptides may be artificially synthesised by coupling the carboxyl group or C terminus of one amino acid to the amino group or N-terminus of another. Generally, either liquid phase synthesis or more commonly, solid-phase synthesis may be employed. An exemplary method for artificial solid phase synthesis involves the use of small porous beads treated with functional units upon which peptide chains can be built. The synthesised peptide usually remains covalently attached to the bead until it is cleaved using a reagent such as anhydrous hydrogen fluoride or trifluoroacetic acid. Automated peptide synthesisers are available, e.g. the automated peptide synthesiser 433A (Applied Biosystems).

Methods for pulsing cells with peptides are well known in the art and any such method can be used to pulse the target cell used in a method of the invention with heterologous peptide. A typical method of pulsing may include incubating cells with peptide in an incubator at a desired temperature (e.g. 37° C.) for a time period, e.g. from 1-5 hours. Particularly, cells e.g. T2 or JY cells, may be cultured, pelleted (by centrifugation) and resuspended in culture medium, e.g. at a concentration of from 1×10⁵ to 1×10⁷, e.g. from 1×10⁶, 2×10⁶, 3×10⁶, 4×10⁶, 5×10⁶ or 6×10⁶. The heterologous peptide may then be added to the cell suspension, where the final concentration of peptide may be from 50-500 μM, e.g. from 100, 200 or 300 μM. Cells may be pulsed at any temperature which does not affect the cell viability, but typically may be pulsed at approximately 20-39° C., e.g. at 37° C. Further, cells may be pulsed for at least 1, 2, 5, 10, or 30 minutes or for at least 1, 2, 3, 4, or 5 hours.

In this embodiment, the method of the invention may comprise an additional step of pulsing the target cells with peptide. Thus, the invention further provides a method of determining the cytotoxic effect of an effector molecule or cell on a target cell presenting a heterologous peptide comprising:
(a) pulsing said target cell with the heterologous peptide;
(b) incubating said effector molecule or cell with said target cell, wherein said target cell is immobilised on a substrate comprising at least one pair of electrodes; and
(c) determining the impedance during and/or after step b), wherein a decrease in impedance is indicative of target cell death.

Although pulsing may be carried out before, during and/or after the target cells are immobilised to the substrate, in a particular embodiment of the invention, the target cells are pulsed prior to immobilisation or attachment to the substrate. As described previously, the inventors have identified that pulsing prior to immobilisation results in a particularly effective assay. In this embodiment, the target cells may be pulsed with the heterologous peptide prior to their introduction to the substrate or prior to being incubated with the substrate. Thus, particularly, the target cells may not be in contact with the substrate before peptide pulsing is carried out. Particularly, the target cells are non-adherent, e.g. non-adherent antigen presenting cells

Thus, in this aspect, the invention further provides a method of determining the cytotoxic effect of an effector molecule or cell on a target cell presenting a heterologous peptide comprising:
(a) pulsing said target cell with the heterologous peptide;
(b) immobilising said target cell on a substrate comprising at least one pair of electrodes;
(c) incubating said effector molecule or cell with said immobilised target cell; and
(d) determining the impedance during and/or after step c), wherein a decrease in impedance is indicative of target cell death.

The heterologous peptide as used in the invention may be any peptide to which an effector is capable of specifically binding. Particularly, the heterologous peptide may be a peptide which is associated with the presence of a disease condition in a patient (e.g. a human patient), and more particularly a peptide which is upregulated in the presence of a disease condition (e.g. where the levels of peptide are increased by at least 10, 20, 30, 40, 50, 60, 70, 80 or 90% as compared to the levels of the peptide in a patient or subject without the disease condition or in the same patient or subject before the disease condition occurred or after successful treatment of the disease condition). Particularly, the heterologous peptide may be only detectable during the disease condition and may not be detectable or may be expressed at very low levels in a patient or subject in the absence of the disease condition. In a particular embodiment, the heterologous peptide may be a tumour associated antigen, which is associated with the presence of one or more types of cancer, e.g. leukaemia, lymphoma, ovarian cancer, pancreatic cancer, bowel cancer, breast cancer, stomach cancer, liver cancer, prostate cancer, brain tumour, skin cancer etc. Thus, the heterologous peptide may be associated with more than one cancer, and may be a generic marker for cancer. The heterologous peptide may, for example, be associated with tumour angiogenesis and thus may be upregulated in the vasculature of many different types of solid tumours. Preferably, the heterologous peptide may be a non-viral tumour associated antigen.

The heterologous peptide may comprise or consist of a fragment of a peptide/polypeptide/protein which is upregulated in a disease condition, such as cancer. Thus, the heterologous peptide may not represent the entire or whole peptide/polypeptide/protein which is upregulated or overexpressed in the disease condition, but may comprise or consist of an antigenic or immunogenic portion of that peptide/polypeptide/protein. Particularly, therefore, the heterologous peptide may represent an epitope of the peptide/polypeptide/protein which is upregulated or overexpressed during a disease condition, e.g. to which an effector cell or molecules may bind. The epitopes of peptides/polypeptides/proteins which are known to be upregulated or overexpressed in disease conditions are generally well known, or can easily be determined using methods known in the art, e.g. by determining antibody binding to synthetic fragments of the proteins.

Particularly, the heterologous peptide may comprise all or an epitope fragment of any of the following tumour associated antigens: CAIX, CEA, CD5, CD7, CD10, CD19, CD20, CD22, CD30, CD33, CD34, CD38, CD41, CD44, CD49f, CD56, CD74, CD133, CD138, a cytomegalovirus (CMV) infected cell antigen, EGP-2, EGP-40, EpCAM, erb-B2, 3, 4, FBP, Fetal acetylcholine receptor, folate receptor-a, GD2, GD3, HER-2, hTERT, IL13R-a2, x-light chain, KDR, LeY, LI cell adhesion molecule, MAGE-AI, MUC1, Mesothelin, NKG2D ligands, NY-ESO-1, oncofetal antigen (h5T4), PSCA, PSMA, ROR1, TAG-72, VEGF-R2, CLEC14A and WT1. Thus, for WT1, the target cell may present the peptide epitope found at amino acid residues 126-134 of WT1 (namely RMFPNAPYL (SEQ ID NO. 1) or the peptide epitope found at amino acid residues 235-243 of WT1 (namely CMTWNQMNL(SEQ ID NO. 2)). For CLEC14A, the target cell may present the whole of CLEC14A or an epitope fragment thereof, for example, residues 97-108 within the C-type lectin like domain of CLEC14A. An advantageous feature of the method of the invention is the ability to express or pulse target cells with the exact specific peptide recognised by a particular effector, e.g. by a T cell having a particular CAR or TCR, demonstrating that the cytotoxic effect is due to real target recognition and not to off target effects.

Although generally the target cell will present a single heterologous peptide, it is also possible for a target cell of the invention to present different heterologous peptides, e.g. at least 2, 3, 4 or 5 different heterologous peptides (which may be from the same or different proteins). In this way, it may be possible to utilise the assay of the invention to determine the effect of an effector which may target (or potentially target) more than one tumour associated antigen on a tumour cell (e.g. an effector cell comprising multiple receptors for different antigens) or of a plurality of effectors which may each target one (or more) of the presented heterologous peptides.

Further, although generally one type of target cell, presenting a single type of heterologous peptide will be used in a method of the invention, it is within the scope of the invention, to employ different target cell types, which may each present the same or different heterologous peptides. Thus, particularly, the method of the invention may be used to determine the cytotoxic effect of an effector or group of effectors, which specifically bind to (or potentially specifically bind to) at least a first and a second heterologous peptide, on a population of target cells comprising target cells expressing a first heterologous peptide and target cells expressing a second heterologous peptide. In this way, it may be possible to use the method of the invention to determine whether dual acting therapies have an appropriate cytotoxic effect against one or more target cells.

As discussed previously, it is possible to vary the amount of heterologous peptide presented (e.g. expressed or displayed) on a target cell (and particularly on a pulsed target cell (e.g. a non-adherent target cell which may be an antigen presenting cell). Thus, it may be desirable to determine the cytotoxic effect on target cells presenting a low amount of heterologous peptide (e.g. pulsed with or expressing a low amount of heterologous peptide), e.g. to determine whether a therapy would have an effect at an early stage of disease, when the peptide may be expressed at lower levels, or to determine whether a therapy would have a cytotoxic effect on non-diseased cells having a low expression level of the peptide. Alternatively, it may be desirable for a target cell to present a high amount of peptide, e.g. to investigate therapy cytotoxic effects at an advanced stage of disease, where peptide expression may be increased. The amount of peptide presented may be controlled by the amount of peptide pulsed or the amount of nucleic acid transduced into a target cell. Thus, if it is desirable for a low amount of peptide to be presented on a target cell, a low amount of peptide may be used for pulsing or a low amount of nucleic acid encoding said peptide may be transduced into a target cell. If a high amount of peptide should be expressed, a large amount of peptide may be pulsed into the target cell or a large amount of nucleic acid encoding said peptide may be transduced into the cell.

To determine a suitable amount of peptide for pulsing or expression, it may be desirable to determine in vivo expression levels of the peptide e.g. in the diseased or non-diseased cell, and to determine what amount of pulsed peptide or transduced nucleic acid encoding the peptide results in an equivalent or comparable expression level in vitro (e.g. an expression level which is at least 70, 80 or 90% identical to that seen in vivo). Peptide expression levels or amounts can be determined by any well-known method in the art, e.g. by immunofluorescence or Western blotting etc.

In this regard, the methods of the invention may comprise a further step of tailoring or matching the amount of heterologous peptide presented by a target cell to that presented by a diseased or non-diseased cell in vivo. As indicated above, in this embodiment, the amount of heterologous peptide presented by a target cell may be at least 60, 70, 80, 90 or 95% identical to the amount presented by a cell in vivo. The methods may further comprise a step of determining the amount of the heterologous peptide that is presented in a cell in vivo.

Additionally, in order to determine the cytotoxic effect of an effector cell or molecule at different stages of disease, it may be desirable to repeat the steps of the method of the invention, using target cells presenting varying or different amounts of heterologous peptide, e.g. with a high amount and with a low amount of peptide. In this aspect, the method may be carried out with target cells presenting a first amount of heterologous peptide and then repeated with target cells presenting a second amount of heterologous peptide, wherein the second amount is higher or lower than the first amount of heterologous peptide, e.g. at least 5, 10, 15, 20, 30, 40 or 50% higher or lower.

The target cells used in a method of the invention may be adherent or non-adherent. “Adherent” cells as used herein refers to cells which are capable of attaching to a substrate or surface without using an intermediate molecule or substance which binds to the cells, allowing their capture. During normal cell culture conditions, adherent cells will grow on a plastic substrate. “Non-adherent” cells as used herein refers to cells which are usually found and/or cultured in suspension and do not generally adhere to a substrate or surface in the absence of an intermediate molecule (capture molecule) or substance allowing their attachment to the substrate or surface. As discussed previously, the method of the invention requires the immobilisation of the target cells to a substrate. It will be appreciated that the mode of immobilisation to the substrate may be dependent on the target cell type used and in particular whether the target cell is adherent or non-adherent.

“Immobilisation” as used herein, refers to the tethering, capture, adherence or binding of the target cells to the substrate. The binding can be direct or indirect (i.e. through another molecule (e.g. an intermediate)) and/or may be reversible or irreversible. For adherent cells, it is possible that immobilisation to the substrate may occur without the use of an intermediate molecule (capture molecule) or substance. However, the binding or immobilisation of adherent cells to the substrate may be enhanced by an intermediate molecule (capture molecule) or substance. Non-adherent target cells, as discussed above, will generally require the use of an intermediate molecule or substance bound to the substrate, which can further bind to the non-adherent cells, in order to be immobilised to the substrate. The intermediate molecule or substance may be itself directly or indirectly (i.e. through one or more other entities) bound to the substrate.

Thus, in one embodiment, the substrate may have a coating to which the target cells bind. The substrate may have a coating over its entire surface or may have a coating only on the substrate surface which in use would be in contact with the target cells. For example, the substrate may be coated over at least 10, 20, 30, 40, 50, 60, 70, 80, or 90% of its surface or of its surface which when in use would be in contact with target cells. When the substrate comprises at least one or more wells, the coating may be present only within the wells of the substrate, e.g. at the bottom of the at least one well(s). It is possible that not all wells comprised within the substrate may be coated, and it is not necessary for the entire surface or the entire bottom of each well to be coated. The coating for example may only be required on the areas of the well which comprise said at least one pair of electrodes or a plurality of electrodes. The coating may improve or enhance the binding of target cells to the substrate, e.g. may reduce the time taken for target cells to bind to the substrate (e.g. by at least 10, 20, 30, 40 or 50%) or may increase the number of cells which bind to the substrate (e.g. by at least 10, 20, 30, 40 or 50%). Particularly, a coating may enhance the binding of adherent cells to the substrate, where adherent cells may be capable of binding to the substrate in the absence of coating.

The coating may comprise a polymer such as a plastic film or one or more biomolecules. The polymer may for example comprise polylysine, polyornithine, peptides or proteins or one or more extracellular matrix components (e.g. fibronectin, vitronectin, laminin, gelatin, collagen (e.g. fibrillar, type I, V and II), glycoaminoglycans or peptidoglycans). Where the substrate is coated with one or more extracellular matrix components, other biomolecules may also be present, including growth factors. Matrigel basement Membrane Matrix (BD Sciences) may be used to coat the substrate of the invention.

The substrate may alternatively or additionally comprise on its surface one or more capture molecules which may specifically bind to a target cell and which may hence immobilise a target cell to the substrate. Such capture molecules may be especially desirable where the target cell is a non-adherent target cell, which may not bind to the substrate in the absence of the capture molecule. The capture molecule may be an antibody, a peptide, polypeptide, or protein, a nucleic acid molecule such as an RNA or DNA molecule or a combination thereof.

The term “antibody”, as used herein, refers broadly to any immunological binding agent or molecule that comprises an antigen binding domain, including polyclonal and monoclonal antibodies. Depending on the type of constant domain in the heavy chains, whole antibodies are assigned to one of five major classes: IgA, IgD, IgE, IgG, and IgM and an antibody capture molecule may be in any one of these classes. Several of these are further divided into subclasses or isotypes, such as IgG1, IgG2, IgG3, IgG4, and the like. The heavy-chain constant domains that correspond to the different classes of immunoglobulins are termed α, δ, ε, γ, and μ respectively. The subunit structures and three-dimensional configurations of different classes of immunoglobulins are well known.

Generally, where whole antibodies rather than antigen binding regions (i.e. antibody fragments) are used as capture molecules in the invention, IgG and/or IgM are preferred because they are the most common antibodies in the physiological situation and because they are most easily made in a laboratory setting. IgG1 antibodies are particularly preferred.

As will be understood by those in the art, the immunological binding reagents encompassed by the term “antibody” extend to all antibodies and antigen binding fragments thereof, including whole antibodies, dimeric, trimeric and multimeric antibodies; bispecific antibodies; chimeric antibodies; recombinant and engineered antibodies, and fragments thereof.

The term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lambda) bodies (scFv-CL fusions); Bispecific T-cell Engager (BiTE) (scFv-scFv tandems to attract T cells); dual variable domain (DVD)-Ig (bispecific format); small immunoprotein (SIP) (kind of minibody); SMIP (“small modular immunopharmaceutical” scFv-Fc dimer; DART (ds-stabilized diabody “Dual Affinity ReTargeting”); small antibody mimetics comprising one or more CDRs and the like.

The techniques for preparing and using various antibody-based constructs and fragments are well known in the art (see Kabat et al., 5th Ed. Public Health Service, National Institutes of Health, Bethesda, Md., 647-669, 1991, specifically incorporated herein by reference). Diabodies, in particular, are further described in WO 93/11161; whereas linear antibodies are further described in Zapata et al. (Protein Eng., 8(10):1057-1062, 1995).

Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art.

The antibodies or antibody fragments can be produced naturally or can be wholly or partially synthetically produced. Thus, the antibody may be from any appropriate source, for example recombinant sources and/or produced in transgenic animals or transgenic plants, or in eggs using the IgY technology. Thus, the antibody molecules can be produced in vitro or in vivo.

The one or more capture molecules may be directly bound to the substrate, or may be bound to the substrate through one or more intermediate molecules. Typically, the capture molecules may be directly bound to the substrate by incubation with the substrate, e.g. for at least 4, 6, 8 or 12 hours, e.g. overnight. Particularly, where the capture molecule is an antibody, a solution having a concentration in the region of 3-6 μg/ml, e.g. at least 4.5 μg/ml may be used, where e.g. at least 10, 20, 30, 40 or 50 μl may be applied to a substrate (e.g. to a well of a 96 well plate). As discussed previously, it may be desirable to immobilise more than one type of target cell to the substrate in the method, which target cells may present different peptides of interest. In this embodiment, it may be desirable for more than one type of capture molecule to be present on the substrate surface. For example, if two different types of target cells should be immobilised to the substrate, it may be necessary to use at least two different capture molecules on the substrate to ensure immobilisation of those cells. However, if the different target cells have any cell marker in common, it may be possible for a single capture molecule to bind both cells types. For example, if target cells are used which are the same target cell type but which express different heterologous peptides, then a single capture molecule may be capable of immobilising both of the target cells expressing different heterologous peptides.

Although it would also be possible to use more than one type of capture molecule on the substrate surface for one type of target cell, e.g. if this enhanced the binding of the target cell to the surface, where the more than one capture molecule (i.e. each capture molecule) is specific for a different marker expressed on the surface of the target cell, it may be preferable to select and use the capture molecule with the highest affinity for the target cell. Although it is possible for the target cell to be bound to the substrate using a capture molecule specific for the heterologous peptide which is presented on the cell surface, it is preferred that the capture molecule binds to an endogenously expressed cell marker or peptide.

As discussed above for the coating of the substrate, the capture molecules may be present across the surface of the substrate or across the surface of the substrate which when in use would be in contact with the target cells. Thus, capture molecules may be present on at least 10, 20, 30, 40, 50, 60, 70, 80 or 90% of the substrate surface or the surface of the substrate which when in use would be in contact with the target cells. Capture molecules may be distributed evenly across the desired surface, or may be placed in concentrated clusters, e.g. upon or across any electrodes which are present within or on the substrate.

It will be appreciated that the choice of capture molecule will largely depend upon the target cell which is to be immobilised to the substrate. However, in a particular embodiment, the capture molecule may be selected from an antibody or an antigen binding fragment thereof which binds to any one more of CD19, CD40, CD22, CD24, CD32a, CD72, CD75, CD77, CD79b, CD83, CD125, CD138, CD139, CD170, CD175, CD307 (a, b, e), CD338 or CD353. IgM may also be used as a capture molecule for particular target cells. Particularly, where antigen presenting cells and more particularly T2 cells are selected as the target cell, it is preferred that the capture molecule is an antibody or antigen binding fragment thereof which binds to CD40. The use of an antibody to CD40 for the immobilisation of non-adherent antigen presenting cells such as T2 cells, has been shown by the inventors to result in a particularly optimal assay, where using an antibody to CD40 for immobilisation results in an enhanced binding of cells, as compared to the use of antibodies which bind to other cell markers. Where it is desired to immobilise non-adherent B cells to the substrate, it may be preferable to use an antibody which specifically binds to CD19 and/or IgM as capture molecules on the substrate.

The “substrate” as used herein refers to any suitable substrate which may comprise or contain at least one pair of electrodes which are needed to measure impedance and to which target cells may be immobilised. The substrate is generally non-conductive (i.e. is not conductive under the conditions under which the substrate is used). The substrate for example may have a resistivity of at least 10⁵ ohm meters, e.g. at least 10⁶ or 10⁷ ohm meters. Substrates for use in the method of the invention may comprise plastic, polystyrene, polyvinyl chloride, polycarbonate, polypropylene, polyester, glass (e.g. lead glass, quartz glass, or borosilicate glass), silicon dioxide on silicon, silicon-on-insulator wafer, ceramics or sapphire. Typically, the substrate or the substrate surface will be biocompatible and thus will not have a detrimental effect on target cells in the method of the invention. Thus, typically, the substrate or substrate surface will not have a cytotoxic or significant cytotoxic effect on the target cells to be used in the method (i.e. typically would not induce cell death in more than 5, 4, 3, 2 or 1% of target cells).

The substrate as used in the invention may be any shape, e.g. may comprise a flat or substantially flat surface, or spherical surface etc. that may be convenient for the immobilisation of target cells and for the subsequent incubation with an effector cell and/or molecule. In a particular embodiment, the substrate may comprise one or more wells or receptacles that may act as one or more fluid containers. In this aspect of the invention it may be possible to immobilise target cells to the bottoms of the one or more wells (e.g. to at least one well) and to carry out one or more methods of the invention simultaneously. For example, where a substrate comprises more than one well, it may be possible to carry out the method simultaneously using different combinations of target cells and effector cells or molecules.

Further, it may be desirable to carry out a control method at the same time as carrying out the method of the invention. Where the substrate comprises more than one well, it may be possible to carry out a control reaction on the same substrate as the method of the invention. A control method may involve carrying out the method with only immobilised target cells (i.e. without the addition or incubation with an effector cell or molecule) and/or may involve using an effector cell or molecule which does not specifically bind to the target cell. Alternatively, a control may involve carrying out the method using only effector cells or molecules without target cell.

Where the substrate comprises one or more wells, the substrate may generally be formed from plastic, glass, or plastic-coated materials such as ceramics, glass or metal etc. Further, the substrate may comprise one or more holes or pores. Preferably, impedance can be determined in the one or more wells, receptacles, holes or pores, and thus the one or more wells, receptacles, holes or pores may comprise electrodes (at least one pair of electrodes e.g. a plurality of electrodes). It is possible that not all of the wells comprised within the substrate may comprise at least one pair of electrodes, but particularly, at least one well will typically comprise at least one pair of electrodes to enable the measurement or determination of impedance.

A typical substrate used in the method of the invention is a multi-well plate, e.g. a microtitre plate. Although, a multi well plate may comprise any number of wells, for example 6, 12, 24, 48, 96, 192, 384, 768 or 1536, a particular substrate for use in the method of the invention is a 96 well microtitre plate.

The substrate may be formed from a single material, e.g. from a single piece of plastic or glass, or may be formed from more than one material and/or may be formed from more than one layer of material e.g. from more than one layer of different materials or the same material. Thus, the substrate may be formed from at least 2, 3 or 4 layers of material.

As discussed previously, the method of the invention requires the detection or measurement of impedance, during and/or after the incubation of an immobilised target cell with an effector molecule or cell, wherein the impedance measurement obtained can be correlated to the number of target cells present and thus to the cytotoxic effect of the effector cell or molecule on the target cell. In order to detect impedance, it is necessary for the substrate to comprise at least one pair of electrodes (e.g. within and/or on the substrate), and preferably for the target cells to be bound to or partially to or between the at least one pair of electrodes. Thus, the binding of target cells to the substrate causes a change in the flow of electrons and thus the current between or across the at least one pair of electrodes present within the substrate and any subsequent change in target cell number (e.g. from cell death) causes a further change in electron flow between the electrodes present. Typically, the more cells bound to or between the at least one pair of electrodes, the greater the reduction in current and the greater the increase in impedance. Alternatively viewed, the less cells bound to or between the at least one pair of electrodes, the greater the increase in current and the greater the reduction in impedance. The change in impedance is indicative and correlates to the number of cells present and specifically of the number of cells bound to and/or between the electrodes.

An “electrode” as referred to herein has a high electrical conductivity and particularly has a higher electrical conductivity than the substrate in which it is comprised, e.g. at least 5, 10, 20, 30, 40, 50, 60, 70, 80 or 90% higher. An electrode may have any structure and may be electrically connected to an impedance analyser. Particularly, an electrode may have a column-like shape or may be spiral.

As discussed above, the substrate comprises “at least one pair of electrodes”. Typically, the at least one pair of electrodes will comprise a first electrode and a second electrode, wherein the first electrode is an anode and the second electrode may be a cathode or earth. The substrate may comprise more than one pair of electrodes, e.g. 2, 3, 4, 5, 10, 20, 30, 40 or 50 pairs of electrodes. Alternatively viewed the substrate may comprise a “plurality” of electrodes. “Plurality” refers to more than one, i.e. at least 2 and more particularly, at least 5, 10, 15, 20, 30, 40, 50, 60, 70, 80, 90 or 100. Thus, it is possible to measure impedance using two electrodes, where those electrodes are constructed to be of a dimension and to be spaced such that an electrical field may be generated between and/or around the electrodes (e.g. a pair of electrodes comprising an anode and a cathode or earth). In a particular embodiment, where a plurality of electrodes are used, the electrodes may be interdigitated with one another (i.e. may have projections that fit into or alongside opposing projections). It will be appreciated that the number of electrodes used in a substrate will be dependent on the size of the substrate and on the surface area of the substrate which in use would be in contact with target cells. As discussed previously, although the plurality of electrodes may be evenly distributed across the surface of the substrate, it is also possible that the plurality of electrodes may be concentrated in particular areas of the substrate, e.g. the areas which in use would be contact with the target cells. Thus, for example, where the substrate is a multi-well plate, the plurality of electrodes may only be placed within at least one of the wells present.

Although, as indicated above, electrodes are distributed across the substrate or a portion of the substrate, it will be appreciated that the plurality of electrodes may comprise spaces or gaps between each electrode or group of electrodes. The spacing between electrodes may be selected to maximise as far as possible the accuracy of the impedance measurement. Thus, in one embodiment, the space between the electrodes may not be significantly greater than the target cells which are used in the method. In this way, the possibility of cell contact with the substrate but not with an electrode or part of an electrode is minimised. Additionally, the space between the electrodes may not be significantly smaller than the target cells which are used in the method. This reduces the chance of a single cell contacting two neighbouring electrodes which could result in an inaccurate impedance signal being generated. Particularly the space between electrodes may be from 0.05 to 3 times the width of an electrode.

The electrode width may also be selected to reduce electrical resistance. Particularly therefore, the width of an electrode may not be too narrow since resistance will increase as the width of the electrodes decreases. An increased resistance may not be desirable since it may cause an electrical potential difference between different points along the electrode, which may result in different impedance measurements for cells which are attached to different parts of the electrode. Where the target cells are eukaryotic cells, such as mammalian cells, an electrode may be greater than 3, 4, 5, 6, 7, 8, 9 or 10 μm wide. However, the width of an electrode is also limited by differences in impedance which may occur for cells which are bound to the centre of an electrode compared to cells bound to the edge of an electrode where the electrical field strength may be greater. Taking this into account, an electrode may ideally have a width which is from 0.5 to 10 times the width of the target cell to be used in the method. Further, preferably all of the electrodes within a substrate, or within a single well of a substrate may have the same width and/or shape.

An electrode may be fabricated from any electrically conductive material, but particularly may be fabricated from copper, aluminium, nickel, chromium, indium tin oxide, gold, silver, steel, and/or platinum. Thus, an electrode may comprise a single or more than one material.

An exemplary substrate comprising at least one pair of electrodes which can be used in the present invention is the xCELLigence Real-Time Cell Analysis (RTCA) system (Acea Biosciences, Inc), which is based on a multiwell plate, where at least one well comprises a plurality of gold electrodes for measuring and/or detecting changes in impedance in a target cell population. Other systems for measuring impedance are also commercially available e.g. Ibidi ICIS impedance culture plates and the ECIS instruments and cultureware available from Applied Biophysics. Any of these systems can be employed in the method of the invention to determine impedance.

The method of the invention requires the incubation of immobilised target cells with effector molecules and/or cells and the measurement of impedance during and/or after the incubation. Measurement of impedance during the incubation (e.g. at one or more time intervals) provides a real time analysis of impedance during the incubation step, which can provide information regarding the timing and duration of any cytotoxic effect that may occur as a result of the incubation. Thus, whilst measurement of impedance after the incubation step may indicate the overall cytotoxic effect of the effector cells and/or molecules on the target cells, the additional measurement of impedance during the incubation can provide further information regarding how that effect occurred. It may therefore be desirable in certain circumstances to measure impedance during the incubation of target cell and effector cell/molecule, as well as at the end of the incubation step.

Further, it may be desirable to measure impedance before immobilisation of the target cells to the substrate. In this way it may be possible to detect or determine the level of target cell immobilisation to the substrate by detecting or measuring impedance, where an increase in impedance would be indicative of target cell immobilisation.

The incubation step may be of any duration, e.g. from 5, 10, 20, 30, 40, 50 or 60 minutes, or from 1, 2, 5, 10, 12, 24, 36, 48, or 72 hours. Particuarly, it may be desirable to incubate for as long as possible in order to determine whether any cytotoxic effect seen is permanent and/or long lasting as opposed to transient e.g. to incubate for at least 4, 5, 6, 7, 8, 9 or 10 days. The incubation step may not be specifically terminated but a final measurement of impedance may be used as a set end point. Alternatively, the incubation step may be terminated by removal of the effector cells/molecule from the substrate to which the target cells were/are bound.

The incubation step may take place at any temperature which does not affect or which does not significantly affect the viability of the target cells or of any effector cells used and which does not affect or significantly affect the function of the effector cells and/or molecule. Particularly, the incubation step may take place at a temperature of from 20-40° C., e.g. at a temperature of 37° C.

Target cells are immobilised to the substrate as described above, e.g. by incubating the target cells with an appropriate substrate (e.g. with any necessary coating or capture molecule) for a sufficient amount of time to allow target cells (e.g. at least 10, 20, 30, 40, 50, 60, 70, 80 or 90% of target cells) to bind e.g. for at least 1, 2, 3, 4, 6, 12 or 24 hours. Typically, it may be desirable for the immobilised target cells to cover at least 10, 20, 30, 40, 50, 60, 70, 80, 90 or 95% of the substrate surface upon which they are in contact with. Thus, where a multiwell plate is used as the substrate, the immobilised target cells may cover at least 10, 20, 30, 40, 50, 60, 70, 80, 90 or 95% of the bottom of one or more of the wells of the plate. It will be appreciated that fewer cells may be initially immobilised on the substrate, and that the substrate may be subjected to culture conditions allowing target cell growth and/or expansion prior to carrying out the incubation step of the method of the invention.

An “effector cell or molecule” as used herein is a cell or molecule which is capable of inducing a cytotoxic effect on a target cell only when specifically bound to a heterologous peptide presented by the target cell. Thus, an effector cell or molecule which is not specifically bound to the target cell (through the heterologous peptide) cannot induce a cytotoxic effect on the target cell, e.g. induces less than 5, 4, 3, 2, or 1% death in a target cell population. An effector cell or molecule as used in the method of the invention may not be capable of specifically binding to the heterologous peptide presented by the target cell and thus may not be capable of inducing a cytotoxic effect (and the method of the invention may be used to identify such effectors). Alternatively, an effector cell or molecule may be capable of specifically binding the heterologous peptide and thus may induce a cytotoxic effect on the target cell. Thus, the methods of the invention may determine whether an effector cell or molecule is capable of binding to the heterologous peptide pulsed or expressed by the target cell. It will be appreciated that an effector cell or molecule which specifically binds to a heterologous peptide presented by the target cell may not necessarily induce cell death in that target cell, although in a particular embodiment, the specific binding of an effector cell or molecule to the heterologous peptide presented by the target cell may be correlated with target cell death (i.e. binding of the effector cell or molecule in most cases will induce cell death e.g. in at least 50, 60, 70, 80, 90 or 95% of target cells). Further, particularly, the effector cell or molecule may not bind to a target cell which is not presenting a heterologous peptide.

In this way, the method of the invention may be used to determine whether a particular effector molecule or cell has a cytotoxic effect (which is usually indicative of specific binding to the target cell through the heterologous peptide) and/or may be used to assess effectors which are known to specifically bind to the heterologous peptide presented by the target cell.

Alternatively viewed, the present invention further provides a method of determining the binding of an effector cell or molecule to a target cell presenting a heterologous peptide (or to a heterologous peptide presented by a target cell), wherein said target cell is immobilised to a substrate comprising at least one pair of electrodes, comprising a) incubating said effector cell or molecule with the immobilised target cell and b) measuring the impedance during and/or after step a), wherein a decrease in impedance is indicative of a cytotoxic effect of the effector cell or molecule and thus of effector cell or molecule binding to the target cell (or heterologous peptide).

Additionally, the invention further provides a method of assessing the affinity and/or avidity of an effector cell or molecule for a heterologous peptide presented by a target cell wherein said target cell is immobilised to a substrate comprising at least one pair of electrodes comprising a) incubating said effector cell or molecule with the immobilised target cell and b) measuring the impedance during and/or after step a) wherein a decrease in impedance is indicative of a cytotoxic effect of the effector cell or molecule and thus of the effector cell or molecule having an affinity and/or avidity for the heterologous peptide. This method may be carried out more than once or several times (simultaneously or separately), to assess the affinity and/or avidity of different effector cells or molecules and may include a step of determining the effector cell or molecule with the highest affinity and/or avidity (which will correlate to the greatest decrease in impedance). Typically, T cells having different TCRs may be tested in this way.

In a particular embodiment, “effector cell or molecule” as used herein may refer to a cell or molecule which specifically binds to a heterologous peptide presented by a target cell, where the effector cell or molecule may be capable of inducing a cytotoxic effect on the target cell.

Particularly, an effector molecule may be any antibody or a soluble T cell receptor (TCR) molecule which may or may potentially specifically bind to a heterologous peptide presented by the target cell. Any such effector molecule, e.g. antibody or soluble TCR, may be further conjugated to cytotoxic moiety. Thus the effector molecule may have a direct or indirect cytotoxic effect on a target cell (i.e. it may itself have a cytotoxic effect or it may be bound to a further moiety or compound which may be cytotoxic to the target cell once bound thereto via the effector molecule).

As indicated above, an effector cell is a cell which is only capable of inducing a cytotoxic effect when specifically bound to a heterologous peptide presented by a target cell. Thus, an effector cell may comprise appropriate signalling pathways and proteins which may be activated upon binding of the cell to the heterologous peptide presented by the target cell and which may induce cell death in the bound target cell. Particularly, an effector cell may comprise one or more receptors e.g. which specifically bind to the heterologous peptide presented on the target cell. The receptors may be naturally occurring, i.e. endogenous receptors, or the effector cell may have been engineered to express a receptor which specifically binds to or potentially specifically binds to a heterologous peptide presented by a target cell (e.g. by transducing the effector cell with a nucleic acid comprising a nucleotide sequence encoding the receptor, as described below). The receptor may particularly be a TCR or a chimeric antigen receptor (CAR) and an effector cell may comprise either or both of these types of receptor.

The term “nucleic acid sequence” or “nucleic acid molecule” or “polynucleotide” or “nucleotide sequence” as used herein refers to a sequence of nucleoside or nucleotide monomers composed of naturally occurring bases, sugars and intersugar (backbone) linkages. The term also includes modified or substituted sequences comprising non-naturally occurring monomers or portions thereof. The nucleic acid, polynucleotide or nucleotide sequences of the present invention may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid, polynucleotide or nucleotide sequences may be double stranded or single stranded. The nucleic acid, polynucleotide or nucleotide sequences may be wholly or partially synthetic or recombinant.

The nucleic acid molecule comprising a nucleotide sequence encoding a peptide (e.g. the heterologous peptide) or a receptor to be used in the present invention may be comprised within a vector. The vector may for example be an expression vector (e.g. a mRNA expression vector or a viral vector). Possible expression vectors include but are not limited to transposons, cosmids, plasmids, or modified viruses (e.g., replication defective retroviruses, adenoviruses and adeno-associated viruses and lentiviruses), so long as the vector is compatible with the target cell used. Particularly, the expression vector may be a gamma retrovirus, such as that described in Engels et al, Human Gene Therapy, 14:1155-1168, 2003, or Schambach et al, Mol. Ther. 2:435-445, 2000, which are incorporated herein by reference. The expression vectors are “suitable for transformation of a target, effector or host cell”, which means that the expression vectors contain a nucleic acid molecule encoding the peptide or receptor and regulatory sequences selected on the basis of the target/effector/host cells to be used for expression, which are operatively linked to the nucleic acid molecule. Operatively linked is intended to mean that the nucleic acid is linked to regulatory sequences in a manner that allows expression of the nucleic acid.

Suitable regulatory sequences may be derived from a variety of sources, including bacterial, fungal, viral, mammalian, or insect genes. Selection of appropriate regulatory sequences is dependent on the target/effector/host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the target/effector/host cell chosen and the vector employed, other sequences, such as an origin of replication, additional DNA restriction sites, enhancers, and sequences conferring inducibility of transcription may be incorporated into the expression vector.

An example of a promoter that may be used is the EF1a promoter, or the CMV promoter. Further examples of promoters include the SV40 early promoter, mouse mammary tumour virus (MMTV), HIV long terminal repeat promoter, MoMuLV promoter, an avian leukaemia virus promoter, an Epstein-Barr virus immediate early promoter, a Rous sarcoma virus promoter, or a MPSV LTR (as described in Engel et al, supra).

The recombinant expression vectors which may be used to transform target/effector/host cells of the invention may also contain a selectable marker gene that facilitates the selection of target cells transformed or transfected with a recombinant molecule. Examples of selectable marker genes are genes encoding a protein such as neomycin and hygromycin that confer resistance to certain drugs, β-galactosidase, chloramphenicol acetyltransferase, firefly luciferase, or an immunoglobulin or portion thereof such as the Fc portion of an immunoglobulin, preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of recombinant expression vectors of the invention and in particular to determine the effect of a mutation on expression and phenotype. It will be appreciated that selectable markers can be introduced on a separate vector from the nucleic acid of interest.

Recombinant expression vectors can be introduced into target/effector/host cells to produce a transformed target/effector/host cell. The terms “transformed with”, “transfected with”, “transformation”, “transduction” and “transfection” are intended to encompass introduction of nucleic acid (e.g., a vector) into a cell by one of many possible techniques known in the art. The term “transformed target/effector/host cell” or “transduced target/effector/host cell” as used herein is intended to also include cells capable of glycosylation that have been transformed with a recombinant expression vector. Nucleic acids can be introduced into mammalian cells via conventional techniques such as calcium phosphate or calcium chloride co-precipitation, DEAE-dextran mediated transfection, lipofection, electroporation or microinjection. Suitable methods for transforming and transfecting host cells can be found in Sambrook et al., 1989 (supra), and other laboratory textbooks, which are incorporated herein by reference.

A number of viral based systems have been developed for gene transfer into mammalian cells, which may be used in the present invention. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art, e.g. a lentiviral vector such as HIV, SIV or FIV. Particularly, as indicated above, a retroviral vector such as a gamma retrovirus may be used, e.g. MP71.

Further, cell penetrating peptides, for example, amphipathic cell penetrating peptides, e.g. KALA, RAWA etc) may be used to transfect cells.

An effector cell may comprise at least one receptor which specifically binds (or potentially specifically binds) to the heterologous peptide presented by the target cell. The effector cell may comprise more than one type of receptor which specifically binds to the heterologous peptide. Thus, for example, it is possible that the heterologous peptide comprises more than one epitope region and thus that an effector cell may comprise receptors which are capable of specifically binding to each epitope present. Further, if the target cell expresses more than one type of heterologous peptide, it is possible that the effector cell may comprise more than one type of receptor e.g. a receptor which specifically binds to each type of heterologous peptide present on the target cell. Thus, an effector cell may comprise at least one, two, three, four or more receptors, which are each capable of binding to a different heterologous peptide or to a different epitope within a heterologous peptide.

A composition comprising more than one type of effector cell and/or molecule may be used in the invention. Thus, a combination of an effector cell and an effector molecule may be used together. Further, effector cells having different specificities may be used, e.g. more than one, two, three or four effector cell types, each of which are specific for a particular heterologous peptide or for a particular epitope within a heterologous peptide.

As described above, an effector cell used in the method of the invention may comprise a TCR and/or a CAR which specifically binds to (or potentially specifically binds to) a heterologous peptide presented by the target cell.

A “T cell receptor” or “TCR” as used herein refers to a molecule which is capable of being expressed on the surface of T cells and which is capable of binding to a particular MHC or HLA/antigen peptide complex (e.g. presented on the surface of an antigen presenting cell or a tumour cell). Thus, TCRs usually recognise antigens or fragments of antigens when found in a complex with a particular MHC or HLA. A TCR molecule as used herein may comprise two protein or polypeptide chains (e.g. may comprise an alpha TCR chain and a beta TCR chain, or a gamma TCR chain and a delta TCR chain), or the TCR may be a single chain molecule, where the alpha and beta chains or the gamma and delta chains are expressed and comprised within a single protein or polypeptide chain. Single chain TCR molecules are described in Chung et al (1994), Proc. Natl. Acad. Sci. USA, 91, 12654-12658, which is incorporated herein by reference.

Each TCR alpha, beta, gamma or delta chain generally comprises a variable region, wherein each variable region typically comprises at least one complementarity determining region (e.g. two and particularly three complementarity determining regions), which is capable of recognising and binding to the tumour associated antigen peptide/MHC complex. Complementarity determining regions (CDRs) may be separated from each other by one or more framework regions (FRs), and typically a TCR alpha, beta, gamma or delta chain variable region as defined herein may comprise three CDRs and three FRs. Particularly, a TCR molecule as described herein may comprise an alpha chain variable region comprising in N to C terminal order FR1α-CDR1α-FR2α-CDR2α-FR3α-CDR3α and a beta chain variable region comprising in N to C terminal order FR1β-CDR1β-FR2β-CDR2β-FR3β-CDR3β. Further, the alpha, beta, gamma or delta chain may comprise a constant region (e.g. having extracellular and transmembrane domains) or a portion of the constant region (e.g. having only an extracellular domain). Particularly, a TCR comprising two separate alpha/beta or gamma/delta polypeptide chains may comprise chains wherein one or both of said chains have a constant domain (e.g. having extracellular and transmembrane domains). In a particular embodiment, both alpha/beta or gamma/delta TCR chains comprise a variable and a constant domain. Single chain TCRs as used herein may comprise a single constant region, e.g. may comprise an alpha chain variable region, a beta chain variable region and a beta chain constant region, or an alpha chain variable region, a beta chain variable region and an alpha chain constant region, comprised within a single polypeptide chain.

The TCR molecule as comprised within an effector cell as used herein, is generally a membrane bound TCR. (Soluble TCR molecules which may be used as effector molecules in the invention generally comprise a truncated constant region or have no constant region, wherein any truncation is sufficient to remove the transmembrane portion of the constant region. Soluble TCRs lacking a transmembrane portion may be of utility in targeting other molecules to the target cells displaying the heterologous peptide, e.g. tumour associated antigen peptide/MHC complex). Thus, particularly, a TCR molecule as present within the effector cell may comprise a constant region transmembrane domain (e.g. one transmembrane domain or two transmembrane domains, one comprised in each chain).

It will be appreciated that the constant regions of the alpha/beta and gamma/delta chains of TCRs are relatively conserved between TCRs. There are thus only two variant beta constant regions (which are different in only 4 amino acids), a single alpha and delta chain constant region, and three variant gamma constant regions in native TCR molecules. Although a TCR molecule as used herein may comprise any of the native constant regions, particularly, the TCR may comprise one or more modifications to any constant region or a portion thereof which is comprised within the TCR. Modifications which improve the pairing of the TCR chains or which improve the production of a soluble or single chain TCR molecule are particularly preferred.

The TCR molecule as used herein may comprise an additional di-S bond which is not present within a naturally occurring molecule, by the substitution of one or more residues in the alpha/beta and gamma/delta chains with a cysteine residue. In this respect, the substitution of an amino acid with a cysteine residue in the beta chain constant region and the substitution of an amino acid with a cysteine residue in the alpha chain constant region may allow the formation of a non-naturally occurring di-S bond between the substituted cysteine residues which may prevent or reduce mispairing of the alpha and beta chains with endogenous alpha and beta chains in T cells. Particularly, the modification may be made to the extracellular portion of the constant region of both chains comprised within the TCR.

More particularly, a TCR molecule as used herein may comprise a substitution at Thr 48 in the constant region of the alpha chain for cysteine and a substitution at Ser 57 in the constant region of the beta chain for cysteine; a substitution at Thr 45 in the constant region of the alpha chain for cysteine and a substitution at Ser 77 in the constant region of the beta chain for cysteine; a substitution at Tyr 10 in the constant region of the alpha chain for cysteine and a substitution at Ser 17 in the constant region of the beta chain for cysteine; a substitution at Thr 45 in the constant region of the alpha chain for cysteine and a substitution at Asp 59 in the constant region of the beta chain for cysteine; and/or a substitution at Ser 15 in the constant region of the alpha chain for cysteine and a substitution at Glu 15 in the constant region of the beta chain for cysteine.

Thus, the TCR molecule as used herein may have a Thr 48 to cysteine substitution in the alpha chain constant region and a Ser 57 to cysteine substitution in the beta chain constant region. Naturally occurring amino acid sequences for the alpha and beta chain constant regions are set out in SEQ ID Nos 3 and 4, respectively, and thus particularly, the modifications discussed above may be made to the stated positions within these sequences.

Other modifications may be made to the TCR to improve the pairing between the chains. For example, a leucine zipper may be utilised, the chains may be murinized or partially murinized e.g. at least one or two amino acids may be murinized, a TCR-like molecule may be constructed (e.g. by fusing the TCR to CD3 zeta) or an amino acid pair at the interface of the alpha and beta constant regions may be inversely exchanged. Particularly, the amino acid pair which are inversely exchanged interact with each other at their surfaces in the native TCR constant regions of the alpha and beta chains. This interacting amino acid pair may be subjected to mutagenesis such that the amino acid of the alpha chain constant domain is replaced by an amino acid which has a sterically projecting group as compared to the naturally occurring amino acid and the amino acid of the beta chain constant domain is replaced by an amino acid which has a sterically recessed group as compared to the naturally occurring amino acid (or vice versa, i.e. the alpha chain amino acid may be substituted with an amino acid having a sterically recessed group and the beta chain amino acid may be substituted with an amino acid having a sterically projecting group). Amino acids which may have a sterically recessed group as compared to a naturally occurring amino acid may be selected from glycine, serine, threonine, valine and alanine. Amino acids which may have a sterically projecting group as compared to a naturally occurring amino acid may be selected from glutamine, glutamic acid, alpha-methylvaline, histidine, hydroxylysine. tryptophan, lysine, arginine, phenylalanine and tyrosine. Particularly, a glycine residue in the alpha constant region may be substituted with an arginine and an arginine residue in the beta constant region may be substituted with a glycine residue, e.g. a glycine to arginine substitution may be made at position 85.1 in the alpha chain constant region and an arginine to glycine substitution may be made at position 88 in the beta chain constant region (using the ImMunoGeneTics information system (IMGT) nomenclature for the numbering of the TCR constant domains). Thus, the arginine to glycine substitution may occur at position 73 of the beta constant region of SEQ ID NO. 4.

Further, it may be desirable to remove a naturally occurring di-S which occurs between the TCR chains (e.g. between the alpha and beta chains). Thus, an interchain native di-S bond in a TCR may be removed by substituting the cysteine residues involved in the bond e.g. to serine or alanine residues, or by deleting the residues. An additional or alternative modification which may be desirable is the removal or substitution of an unpaired cysteine residue which occurs in the native beta TCR chain. Such a modification may be preferred wherein the TCR is a single chain TCR.

In a particular embodiment of the invention, the TCR molecule may recognise and bind to a WT1 peptide/MHC or HLA complex and more particularly to a WT1 peptide/HLA A2 complex. For example, the TCR may bind to a WT1 peptide 235-243 (CMTWNQMNL) (SEQ ID NO. 2)-HLA A* 2402 combination. However, in a particularly preferred embodiment, the TCR recognises and binds to the RMFPNAPYL (SEQ ID NO. 1) peptide of WT1/HLA A2 complex. It will be appreciated that more than one TCR may be capable of binding to this complex and one or more of such TCRs may be used in an effector cell in the present invention. Further, in connection with this aspect, the effector cell may utilise a double chain TCR (i.e. having separate alpha and beta or gamma and delta chains) and/or a single chain TCR to bind to the WT1 complex.

Thus, a TCR molecule as used in the effector cell described herein may comprise an alpha chain and a beta chain,

wherein the alpha chain comprises CDR1alpha of SEQ ID NO. 5, CDR2 alpha of SEQ ID NO. 6 and CDR3 alpha of SEQ ID NO. 7 or 8, and

wherein the beta chain comprises CDR1 beta of SEQ ID NO. 9, CDR2 beta of SEQ ID NO. 10 and CDR3 beta of SEQ ID NO. 11 or 12,

or a variant thereof wherein one or more of the CDRs comprise one, two or three amino acid substitutions,

wherein said TCR molecule is capable of binding to an HLA A2/RMFPNAPYL complex.

It should be noted that in some nomenclature systems the CDR3 of the beta chains may be defined to be longer than in the nomenclature system used in the Immunogenetics (IMGT) database described below. Additionally, in some nomenclature systems, the CDR3 of the alpha chains may be define to be shorter than in the IMGT system. Similarly, the constant region may or may not include framework residues flanking the CDR3 region in the different nomenclature systems.

Thus, using the IMGT system, CDR3 alpha may have the amino acid sequence of SEQ ID NO. 7 and the constant region includes the framework amino acid sequence FGKGTHLIIQP (SEQ ID NO. 13).

Using a different nomenclature system (Garcia) (Garcia et al, 1999, Ann. Rev. Immunol. 17, 369-397, incorporated herein by reference), CDR3 alpha has the amino acid sequence of SEQ ID NO. 8, the framework region immediately C-terminal to this has the amino acid sequence of FGKGTHLIIQP and the constant region begins with the amino acid sequence YIQ.

Using the IMGT nomenclature system, CDR3 beta may have the amino acid sequence SEQ ID NO. 11 and the constant region immediately C-terminal to this includes the framework amino acid sequence SET.

Using the Garcia nomenclature system, CDR3 beta has the amino acid sequence SEQ ID NO. 12 and the framework region immediately C-terminal to this has the amino acid sequence FGPGTRLLVL (SEQ ID NO. 14) and the immediately C-terminal constant region begins with the amino acid sequence EDL.

It will be appreciated that a skilled person can readily design and synthesise TCRs for use in the present invention, using either or any nomenclature system, provided that the framework region is compatible with the CDRs. The amino acid sequences, including variable regions (and thus framework regions) of numerous TCR alpha and beta chains are well known in the art, some of which are described at IMGT (Immunogenetics) database at http://imgt.cines.fr. This information together with for example, Garcia et al (supra), may be used to design and produce TCRs comprising CDRs and FRs.

As indicated above, variant TCRs may be used in the effector cells in the method of the present invention, where one or more CDRs may comprise one, two or three amino acid substitutions. Particularly the substitutions may be conservative substitutions, and any variant molecules should be capable of binding to the HLA A2/RMFPNAPYL complex. The affinity of binding of the variant may be increased or decreased as compared to the TCR having the CDRs as defined above, and the method of the invention can be used to determine the cytotoxic effect of any such variant on a target cell.

Particularly, the TCR molecule for use in an effector cell in the method of the present invention may comprise a TCR alpha chain as set out in SEQ ID NO. 15 and a TCR beta chain as set out in SEQ ID NO. 16. Alternatively, the TCR molecule may comprise a TCR alpha chain as set out in SEQ ID NO. 17 and a TCR beta chain as set out in SEQ ID NO. 18, wherein said alpha and beta chain sequences maybe further modified to stabilise or to enhance the pairing of said alpha and beta chains.

A “CAR” or “chimeric antigen receptor”, used interchangeably herein, refers to a molecule, which comprises at least three domains, namely an extracellular domain comprising an antigen binding domain (i.e. a domain which may bind specifically to a heterologous peptide presented by a target cell), a transmembrane domain and an intracellular domain comprising an intracellular signalling domain.

Thus, when a CAR is expressed on an effector cell, the antigen binding domain will be present within or as the extracellular domain. Typically, most or all of the antigen binding domain will be present extracellularly, to allow the binding of the CAR to the heterologous peptide (e.g. at least 90, 95, 97, 99 or 100% of the antigen binding domain will be present extracellularly when the CAR is expressed in an effector cell, transported to the cell membrane and presented).

The transmembrane domain links the extracellular domain comprising the antigen binding domain (i.e. the anti-heterologous peptide binding domain) to the intracellular signalling domain and typically spans the cell membrane of a host cell after CAR expression and membrane targeting. Thus, the transmembrane domain passes through the cell membrane after CAR expression and membrane targeting. The transmembrane domain may be derived from or based upon a protein having at least one transmembrane domain and/or extracellular and/or intracellular portions and thus the transmembrane domain of a CAR may be attached at the N and/or C termini to extracellular and/or intracellular sequence/polypeptide/protein from the protein from which it is derived or based upon. Thus, when the transmembrane domain is obtained or derived from a known transmembrane protein, additional sequence may be present extracellularly and/or intracellularly, together with the transmembrane domain which passes through or spans the membrane, to attach the CAR thereto. As discussed further below, the transmembrane domain may be derived from a protein or a portion of a protein which has both transmembrane and intracellular regions, e.g. CD28, and both of these domains or portions thereof maybe comprised within a CAR which is present in an effector cell used in the method of the invention.

The intracellular signalling domain of the CAR, is present within the effector cell (i.e. is comprised within the intracellular domain of the CAR) after expression of the CAR, typically within the cytoplasm of the cell. This domain is capable of activating one or more normal functions of the effector cell in which the CAR is expressed. For example, if the effector cell is a T cell, then the intracellular signalling domain may be capable of activating the cytolytic or helper activity of the T cell. CARs may additionally comprise further domains e.g. at least one co-stimulatory domain such as 41BB, OX40, CD28 etc. In a particular embodiment, the CAR expressed on the effector cell may be capable of binding to CLEC14A or to a portion of CLEC14A.

An effector cell for use in the method of the invention may be an immune cell, particularly a mammalian immune cell, such as a human immune cell. Immune cells are capable of having an effector function and include T cells and NK cells. The T-cell may be any type of T-cell, including an alpha-beta T cell, a gamma-delta T cell, a memory T cell (e.g. a memory T cell with stem cell-like properties). The NK cell may be an invariant NK cell.

The effector cell for use in the method of the invention may be purified (i.e. may be specifically isolated before use) or may be non-purified (e.g. used in a composition where other cells or components may be present e.g. as PBMC).

The term “mammalian” as used herein refers to any mammal, but particularly refers to a human, a domestic animal (e.g. a cat, dog etc), a horse, a mouse, a rat, a primate, such as a monkey, a cow, a pig etc.

The T cells may be obtained from a number of sources, including from peripheral blood mononuclear cells, bone marrow, lymph node tissue, cord blood, thymus tissue, tissue from a site of infection, ascites, pleural effusion, spleen tissue and tumours. Particularly in the present invention, immune or T cells may be obtained from a subject having a condition which may be treated with immune or T cells expressing a receptor which binds to the heterologous peptide (e.g. a tumour associated antigen). T cells (or immune cells) may be obtained by any method known in the art.

T cells may also be obtained “off the shelf” and thus may not necessarily be obtained from a subject having a condition which may be treated with an immune cell comprising a receptor which specifically binds to the heterologous peptide. Thus T cells for use in the present invention may have previously been stored and/or modified prior to transduction with a nucleic acid or vector encoding a receptor which binds to the heterologous peptide (e.g. a tumour associated antigen).

Particularly, T cells may be obtained from a unit of blood (particularly anticoagulated blood) collected from a subject using any suitable techniques in the art such as Ficoll separation. Alternatively, immune cells (e.g. T cells) may be obtained from a subject (typically a mammalian subject) by apheresis, where the apheresis product typically comprises lymphocytes (including T cells, monocytes, granulocytes, B cells, other nucleated white blood cells, red blood cells and platelets). It will be appreciated that cells collected by apheresis may be washed to remove the plasma fraction and to place the cells in an appropriate buffer or media for subsequent processing, e.g. the cells may be washed using PBS, or using a wash solution lacking divalent ions e.g. lacking calcium and/or magnesium. Washing steps may be achieved by methods known in the art e.g. by using a semi-automated “flow-through” centrifuge (e.g. the Cobe 2991 cell processor, the Baxter CytoMate, or the Haemonetics Cell Saver 5). After washing, the cells may be resuspended in a variety of biocompatible buffers, such as for example, Ca-free, Mg-free PBS, PlasmaLyte A, RPMI 1640 (Sigma) or PBS or other saline solution with or without buffer. Typically, in the UK, cells for transfusion are treated in accordance with the guidelines found at http://www.transfusionguidelines.org.uk/red-book. Further procedures acceptable in Europe may be found in the Guide on the preparation, Use and Quality assurance of Blood Components, Current edition, EDQM. In the US, typically the AABB Blood and Blood components guidelines are followed. The WHO requirements also exist for the collection, processing QC of blood, blood components and plasma derivatives.

T cells may be isolated from peripheral blood lymphocytes by lysing red blood cells and depleting the monocytes, e.g. by centrifugation through a PERCOLL™ gradient or by counter-flow centrifugal elutriation. It is possible to select specific populations of T cells for use in the present invention. However, selection is not compulsory and a mixed population of cells (e.g. comprising different types of T-cells) may be transduced with a nucleic acid or vector encoding a receptor as described above. Subpopulations of cells which may be selected include CD3+, CD28+, CD4+, CD8+, CD45RA+ and CD45RO+ T cells. Selection of particular populations may be achieved using beads coupled with antibodies which selectively bind to antigens expressed on T cells populations. A combination of antibodies directed to surface markers uniquely expressed in particularly T cell populations may be used for selection.

T cells may also have been derived (differentiated) from iPS cells or stem cells, e.g. embryonic stem cells.

Any amount of effector cell or molecule may be incubated with an immobilised target cell in the method of the invention. However, as it will be appreciated that for a medical purpose, it is desirable to use as little active as possible (e.g. effector cell or molecule), it may be advantageous to use the method of the invention to investigate different amounts of effector cell or molecule which may result in a cytotoxic effect of the target cells. This may provide information regarding the minimal amount or dose of effector which may be needed to provide the desired cytotoxic effect. Particularly, for example, in a medicament, it may be desirable or only possible to administer effector cells in a ratio of at most 10:1 to 1:100 (effector cell:target cell), e.g. from 5:1, 4:1, 3:1, 2:1 or 1:1. Such a ratio of effector cells may therefore be used in the present invention.

The method of the invention requires the measurement or determination of impedance to determine whether the effector cell or molecule has a cytotoxic effect on the target cell presenting a heterologous peptide. In one embodiment, it may be desirable to additionally measure or determine the impedance before addition or incubation of the effector cells with the target cells (i.e. the impedance of the substrate with immobilised target cells), in order to compare the impedance before and after (or during) the incubation with effector cells. A change in impedance (and particularly a decrease of impedance) during or after the incubation with effector cells as compared to before incubation with effector cells (i.e. when only target cells are present and immobilised to the substrate) may be indicative of a cytotoxic effect. For a standardised method, where impedance values are already correlated to a cytotoxic or no cytotoxic effect, it may not be necessary to carry out an initial measurement or determination of impedance, before the incubation with effector cells or molecules.

Further, as discussed previously, it may be desirable to measure impedance during incubation with the effector molecule and/or cells. Any number of measurements may be taken during the incubation to provide an indication of the real time cytotoxic effect of the effector cells and/or molecule. For example, a measurement may be taken at set intervals e.g. approx. every 10 seconds, approx. every minute, approx. every 5 mins or approx. every 10 mins, or at longer intervals, e.g. approx. every 30 mins, 1-2 hours, 6-12 hours, 24-48 hours etc. Thus, it may be desirable to measure impedance at least once during incubation with effector cells and/or molecules, e.g. at least 2, 3, 4, 5, 10 or 20 times.

As indicated above, a decrease in impedance may be indicative of a cytotoxic effect of the effector cells on the immobilised target cells. Particularly a decrease in impedance of at least 5, 10, 15, 20, 30, 40, 50, 60, 70, 80, 90 or 95% may be indicative of a cytotoxic effect, e.g. as compared to the impedance measurement prior to incubation with an effector cell and/or molecule (e.g. an impedance measurement when the target cells are immobilised to the substrate but prior to incubation with effector). The decrease in impedance is particularly a decrease that can be detected by an impedance analyser e.g. by using a resistor. Impedance as used herein refers to the electrical or electronic impedance.

Impedance may be measured in the present invention by applying a voltage at at least one frequency (e.g. at a single or multiple frequencies) and by monitoring the current through the electrodes. Impedance may be calculated by dividing the voltage amplitude by the current amplitude (using the voltage and current values obtained at the same frequency). Alternatively, impedance may be measured by applying an electric current at any frequency (single or multiple frequencies) through the electrodes and determining the resulting voltage, where impedance is calculated by dividing the voltage by the current (values from the same frequency).

Impedance in the present invention is correlated with the number of target cells present and thus cell death will affect (decrease) the impedance. A decrease in impedance upon addition or incubation with effector cells and/or molecules is thus indirectly indicative of the cytotoxic effect of the effector cell or molecule. Thus, if the effector cell and/or molecule has a cytotoxic effect on the target cells (i.e. is capable of inducing and/or causing cell death of the target cells), a decrease in impedance may be detected. Particularly, death of at least 1, 2, 5, 10, 20, 30, 40, 50, 60, 70, 80 or 90% of the target cells may result in a decrease in impedance. Generally, the more target cell death induced by the effector cell and/or molecule the greater the decrease in impedance which is detected. As previously discussed, if the effector cell and/or molecule does not induce a cytotoxic effect on the target cells, then no change in impedance may be detected or an increase in impedance may be detected relating to target cell growth or expansion. The method of the invention may comprise an additional step of determining the cytotoxic effect.

The method of the invention may be used not only to determine whether an effector cell and/or molecule has a cytotoxic effect on a target cell, but may be used to determine the level of the cytotoxic effect in relation to time (i.e. how quickly the cytotoxic effect took). Further, the method may be used to determine whether that effect is long lasting or persistent. The method of the invention can be used as an initial screen to determine whether a therapy has any efficacy against its target or even to determine whether a patient's own cells once modified to express a particular receptor, are capable of inducing a cytotoxic effect, before a therapy is given to the patient.

In a particular embodiment, the invention provides a method of determining the cytotoxic effect of an effector cell on a target antigen presenting cell presenting a heterologous peptide comprising:
(a) incubating said effector cell with said target antigen presenting cell, wherein said target antigen presenting cell is immobilised on a substrate comprising at least one pair of electrodes; and
(b) determining the impedance during and/or after step a), wherein a decrease in impedance is indicative of target cell death.

More particularly, the antigen presenting cell may be a non-adherent antigen presenting cell, e.g. a T2 antigen presenting cell, which is immobilised to the substrate using an antibody which binds to CD40. Further, in this particular embodiment, the heterologous peptide may be WT1 or a epitope therefrom, particularly a peptide comprising or consisting of SEQ ID NO. 1, and the effector cell may be a T cell which comprises a TCR which specifically binds to WT1 or to an epitope therefrom e.g. to SEQ ID NO.1.

As discussed previously, in a particular embodiment of the invention, target cells (particularly non-adherent target cells) are pulsed with peptide prior to immobilisation to the substrate. This has been identified by the inventors to result in a beneficial and improved cell index (i.e. an increased number of target cells which adhere or are immobilised to the substrate comprising at least one pair of electrodes). Increases of at least 10, 20, 30, 40 or 50% of target cell adherence to the substrate may be achieved by pulsing with peptides prior to attachment, rather than after attachment. The cell index can be determined by immuno-staining target cells and/or manual counting by microscopy.

In this embodiment, the invention provides a method of determining the cytotoxic effect of an effector molecule or cell on a non-adherent target cell, particularly a non-adherent antigen presenting cell, presenting a heterologous peptide comprising:
(a) pulsing said target cell with the heterologous peptide;
(b) immobilising said target cell on a substrate comprising at least one pair of electrodes;
(c) incubating said effector molecule or cell with said immobilised target cell; and

determining the impedance during and/or after step c), wherein a decrease in impedance is indicative of target cell death.

Alternatively viewed, the invention provides a method for increasing the cell index of non-adherent peptide pulsed target cells on a substrate comprising at least one pair of electrodes, comprising pulsing said cells with peptide prior to attachment to said substrate.

The present invention further provides the use of impedance to determine the cytotoxic effect of an effector cell and/or molecule on a target cell presenting a heterologous peptide to which the effector cell and/or molecule specifically bind. Particularly, the target cell is immobilised to a substrate comprising at least one pair of electrodes as previously described.

The invention further provides a kit for use in a method of the invention, wherein said kit comprises:
(a) a target cell presenting a heterologous peptide or a target cell and a heterologous peptide or a nucleic acid comprising a nucleotide sequence encoding said heterologous peptide;
(b) a capture molecule for immobilising said target cell to a substrate;
(c) instructions for use of (a) and (b) in a method of the invention; and optionally
(d) a substrate comprising at least one pair of electrodes; and/or
(e) an effector molecule and/or cell which specifically binds to the heterologous peptide presented by the target cell, or an effector cell and a nucleic acid comprising a nucleotide sequence encoding a receptor which specifically binds to the heterologous peptide presented by the target cell.

The invention will now be described in terms of the figures and non-limiting examples as set out below:

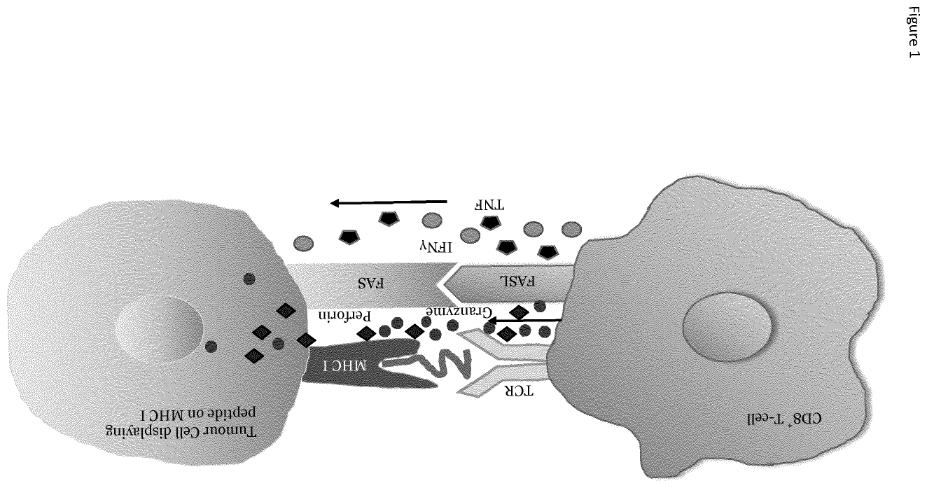

FIG. 1 shows the mechanisms involved in cytotoxic T lymphocyte (CTL) and natural killer (NK) mediated target cell killing.

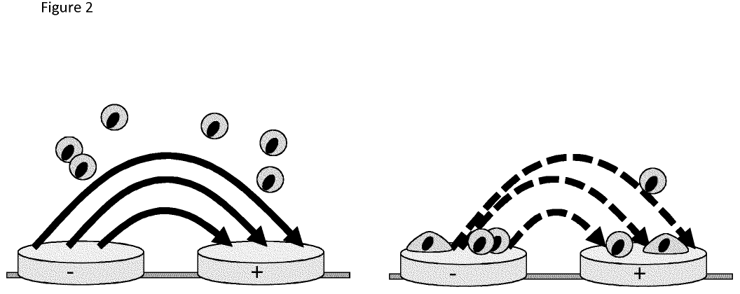

FIG. 2 shows diagrammatically how the presence of cells on a substrate can impede electron flow.

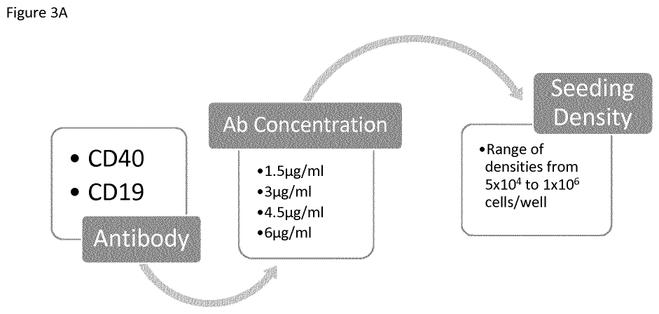

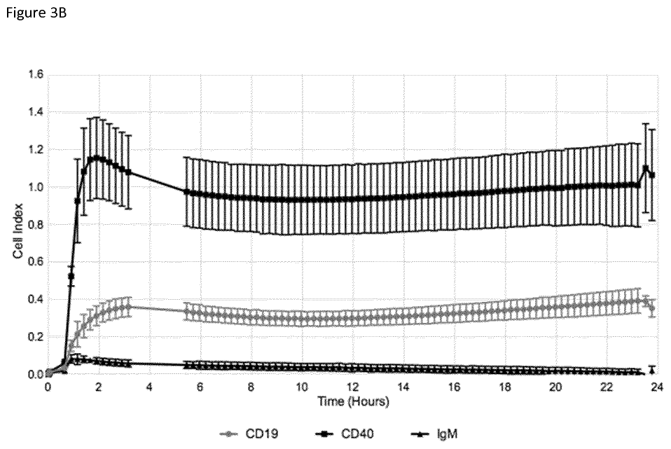

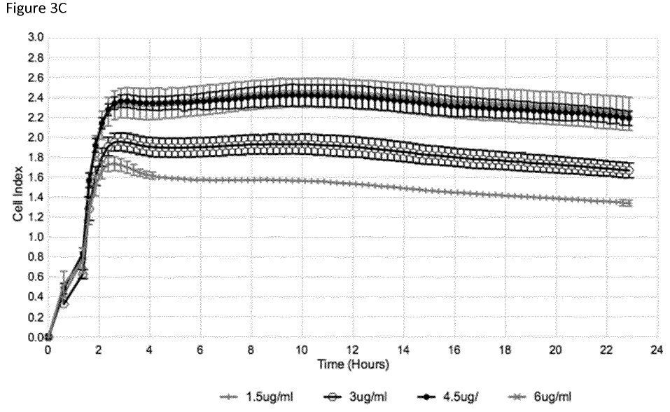

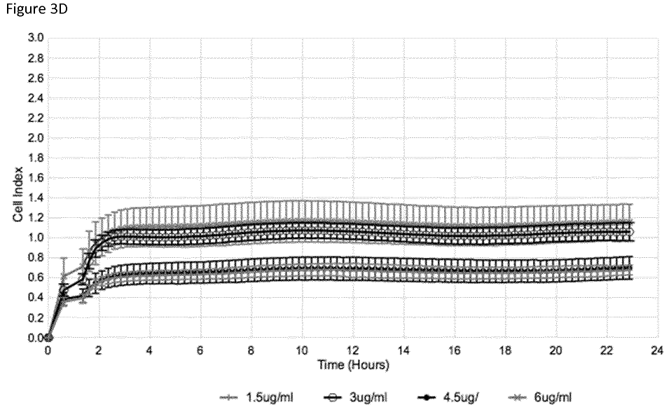

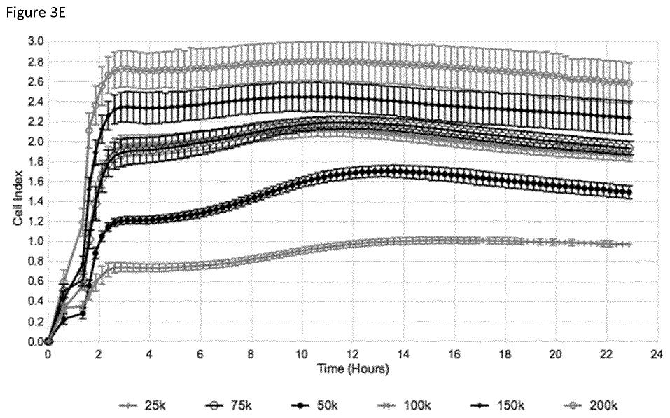

FIG. 3 shows the optimisation of antibody concentration for non-adherent cell immobilisation and of cell seeding density for an impedance assay using WT1 pulsed APC (T2 cell line). FIG. 3A shows the amounts of antibodies and cells used. FIG. 3B shows the change of cell index (CI) on E-plates coated with 6 μg/ml of CD19 and CD40 and 1.5 μg/ml of IgM (50000 T2 cells). CD40 shows the highest increase of CI. CD19 shows moderate attachment and IgM showed a poor increase of CI. FIG. 3C shows the cell attachment impedance plot for CD40 using 100000 cells/well. 1.5 μg/ml coating had the lowest binding efficiency, 3 μg/ml showed a higher CI and there was no difference in the maximum CI between 4.5 and 6 μg/ml. FIG. 3D shows cell index changes when using gradients of concentration of anti CD19 antibodies. FIG. 3E shows the optimisation of cell seeding density when using 6 μg/ml of anti-CD40 antibody.

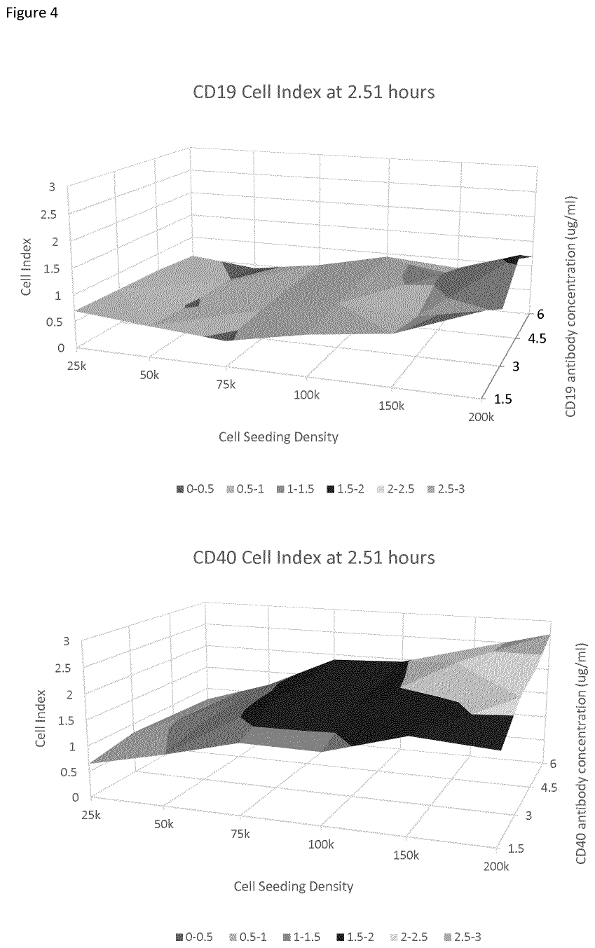

FIG. 4 shows the optimisation results for seeding density and antibody concentration, demonstrating the advantageous effect of using an anti-CD40 antibody for immobilisation.

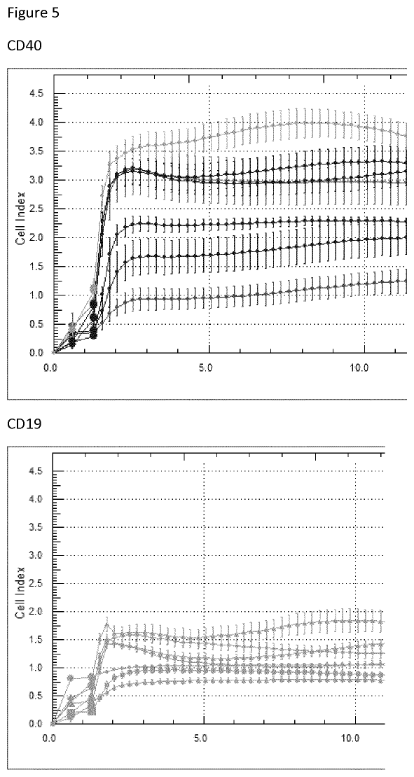

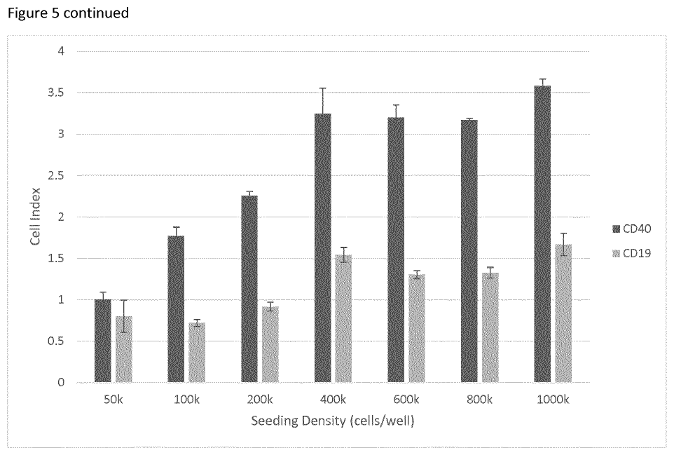

FIG. 5 shows the cell density required for maximum cell index, using either anti-CD40 or anti-CD19 antibodies. The bar chart shows the seeding density on the x axis, where the left hand bar at each cell density measurement shows the CD40 cell index and the right hand bar at each cell density measurement shows the CD19 cell index.

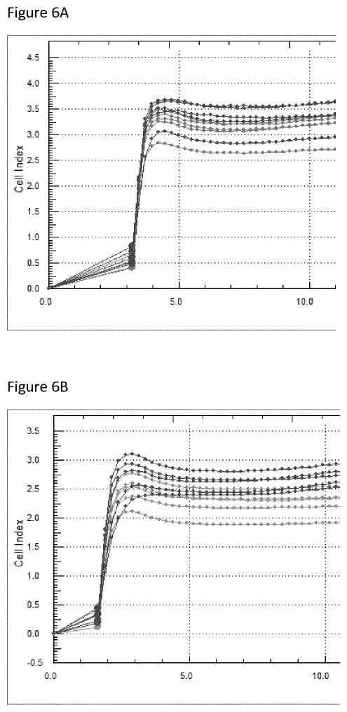

FIG. 6 shows the effect of peptide pulsing on cell index, where cells pulsed prior to attachment result in a higher cell index. FIG. 6A shows pulsing before attachment and FIG. 6B shows pulsing after attachment.

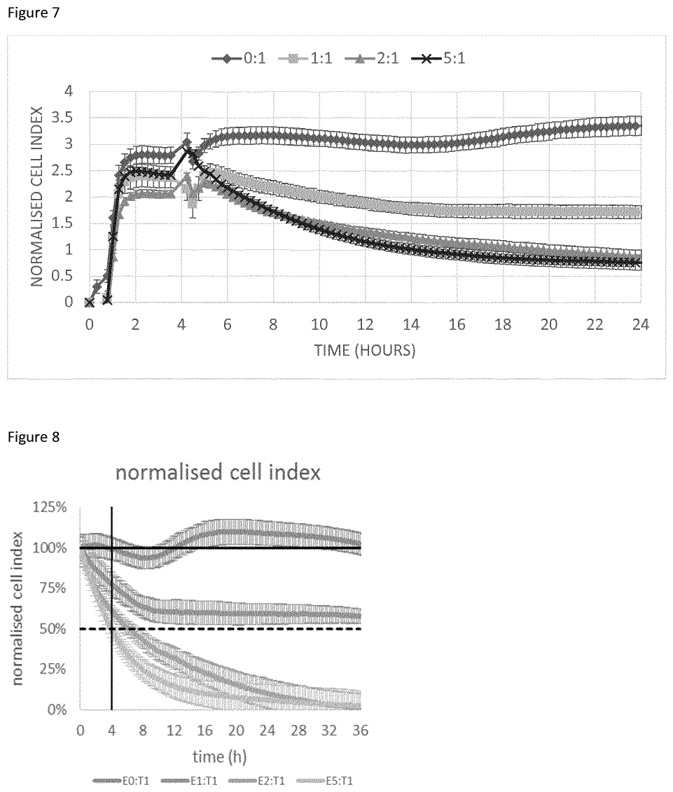

FIG. 7 shows the optimisation of effector:target ratios for T2 cells pulsed with SEQ ID NO. 1 and for WT1 effector cells added after 4 hours of target cell attachment. The top line shows a 0:1 ratio, the second line from the top shows a 1:1 ratio, the third line from the top shows a 2:1 ratio and the line at the bottom shows a 5:1 ratio.

FIG. 8 shows a normalised WT1 potency assay using impedance. When using an effector:target ratio of E1:T1, the killing stops at 8-12 hours. 50% cell death is observed for effector:target ratios of 2:1 and 5:1 and EC50 is reached within 4 hours for an effector:target ratio of 5:1.

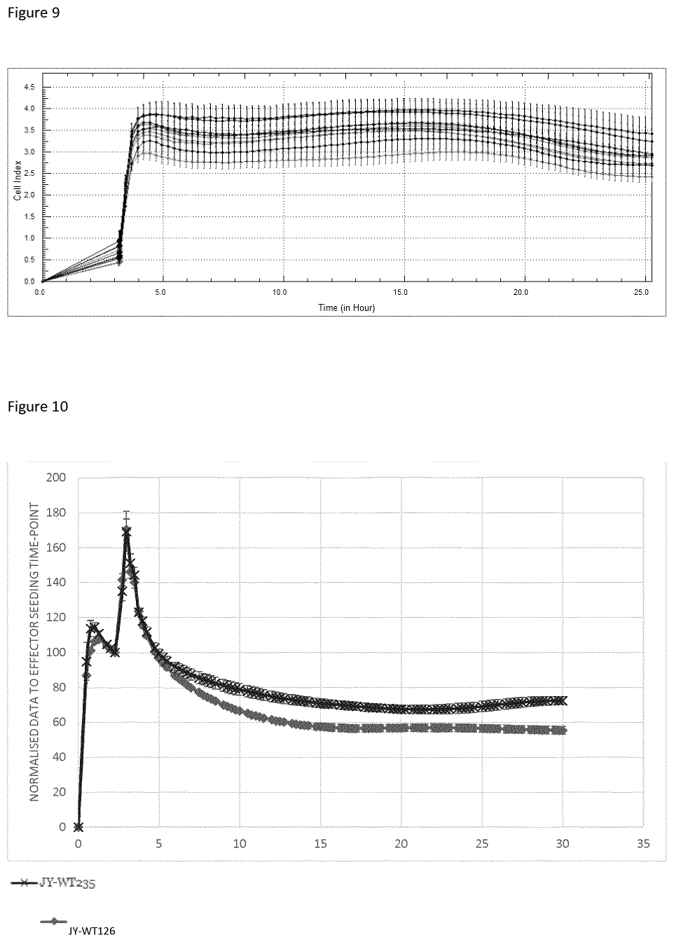

FIG. 9 shows there is no difference in T2 attachment in cells loaded with either WT126 or WT235.

FIG. 10 shows the killing of JY target cells which have been pulsed with WT126 as opposed to JY cells pulsed with WT-235, by effector cells.

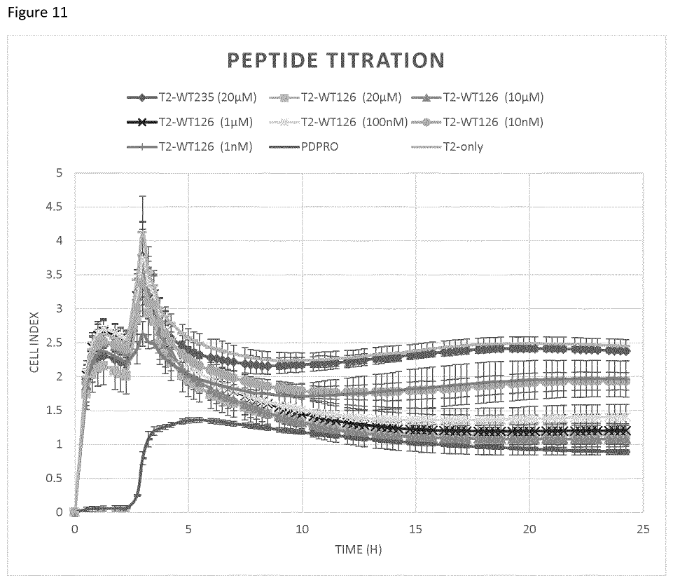

FIG. 11 shows a peptide titration experiment in T2 cells, were different amounts of peptide was pulsed into the cells. The experiment demonstrates that the amount of peptide presented can be tailored by pulsing with different peptide amounts.

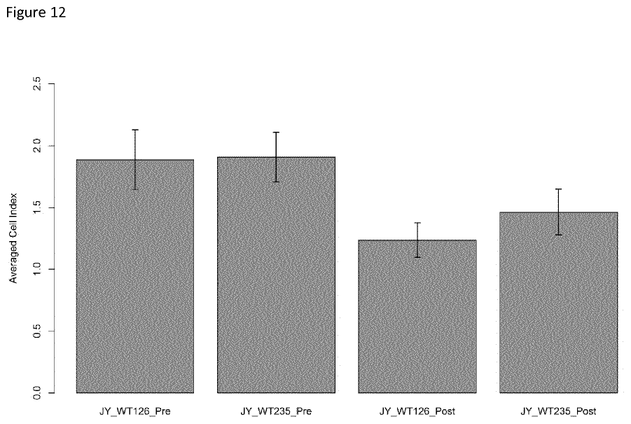

FIG. 12 shows a bar chart demonstrating the killing of JY cells pulsed with WT126 as opposed to WT235. Bar chart shows the results for the JY cell line before (Pre) and after (Post) treatment with WT-1 transduced cells. The height of the bars represents the mean across all replicates per peptide and time point. Error bars denote confidence intervals of the standard error of the mean (SEM) at 95% probability level. A post-hoc t-test demonstrated significant differences between WT 126 and WT 235 after the induction of WT-1 transduced cells (JY_WT126_Post vs. JY_WT235_Post, P=0.023).

### EXAMPLES

### Example 1: Antibody Mediated T2 Cell Attachment

### Materials and Methods

### T2 Cell Culture

T2 cells were obtained from ATCC. Cells were cultured in RPMI 1640 supplemented with Glutamax (ThermoFisher) containing 20% foetal bovine serum (FBS) at a seeding density of 0.5×10⁶ cells/ml. Cells were cultured for a minimum of 4 days before experimentation.

### Attachment Substrate

Attachment substrate was prepared by making a 6 μg/ml solution of anti-CD40 and anti-CD19 (both from R&D) in PBS (Sigma). IgM attachment substrate was prepared by making a 1.5 μg/ml solution of IgM (Cambridge Biosciences) in PBS. 50 μl of each substrate solution was added to each well of a 96-well E-Plate 96 (ACEA Bioscience) and the lid replaced and secured with parafilm. The plate was placed into a 4° C. fridge for 12-18 hours.

### Cell Seeding

Attachment substrate was aspirated and the wells were washed three time using PBS. Cultured T2 cells were seeded at 50,000 cells/well. The plate was placed on the xCELLigence RTCA MP (ACEA Bioscience) module and sweeps were performed every 15 minutes for 24 hours.

### Results

### CD19, CD40 and IgM Mediated T2 Cell Attachment

First, the individual antibodies were compared for their binding efficiency. FIG. 3B shows the change of cell index (CI) on E-Plates coated with 6 μg/ml of CD19 and CD40, and 1.5 μg/ml of IgM. Each well was seeded with 50,000 T2 cells. CD40 shows the highest increase of CI with a maximum of 1.15±0.22 at 1.8 hours following attachment. CD19 shows moderate attachment with a maximum of 0.36±0.05 after 3 hours of attachment. In contrast, IgM showed poor increase of CI with a maximum of 0.08±0.018. From these results, CD19 and CD40 were chosen for the remaining optimisation experiments.

### Example 2: T2 Cell Attachment Optimisation

### Materials and Methods

### As Above

### Results

### Antibody Concentration

Anti-CD40 and anti-CD19 were coated in increasing concentrations from 1.5 μg/ml, 3 μg/ml, 4.5 μg/ml and 6 μg/ml on the E-Plate 96. FIG. 3C shows the cell attachment impedance plot for CD40 using 100,000 cells/well over 24 hours. The increase of cell index (CI) over the first 3 hours shows the binding of the T2 cells onto the surface of the E-Plate. The graph shows a coating concentration of 1.5 μg/ml had the lowest binding efficiency with a maximum CI of 1.35±0.067. The CI of 3 μg/ml coated-wells showed a higher CI of 1.7±0.14. There was no difference in the maximum CI between the 4.5 μg/ml and 6 μg/ml coated wells at 1.90±0.14 and 2.06±0.13 respectively.

FIG. 3D cell index changes when using gradients of concentration of anti-CD19 antibody. There was no significant difference between well coated with 3 μg/ml or 6 μg/ml where the CI was 1.15±0.09 and 1.04±0.20.

### Cell Seeding Concentration

The effect of cell seeding was then determined. FIG. 3E shows the plot of various seeding densities using anti-CD40 (6 μg/ml). Under these conditions, the maximum cell index increased proportionally to the increasing seeding density, this ranged from a maximum CI of 1.01±0.03 in wells seeded with 25,000 T2 cells to 2.8±0.20 in wells seeded with 200,000 cells.

### Effect of Antibody Concentration and Seeding Density on Cell Index

A range of both anti-body coating concentration and cell seeding densities were examined together to demonstrate the relationship between the two factors on CI. E-plates were coated with anti-CD40 and anti-CD19 at 1.5 μg/ml, 3 μg/ml, 4.5 μg/ml and 6 μg/ml for 16 hours before being seeded with 25K, 50K, 75K, 100K, 150K or 200K cells/well. Surface plots for anti-CD19 and anti-CD40 are shown in FIG. 4 where the CI was taken after cell attachment at 2.51 hours. With both antibodies there was a linear relationship between coating concentration and seeding density.

### Determining Maximum Seeding Concentration

As seen in FIG. 4 the maximum saturation concentration of CD40 is 4.5 μg/ml. To obtain the highest CI the seeding densities was increased to 50 k, 100 k, 200 k, 400 k, 600, 800 k and 1000 k cells/well. FIG. 5 shows the CI comparing CD19 and CD40 at 2.5 hours after seeding the T2 cells. In all densities, except 50 k cells/well, wells coated with anti-CD40 had significantly higher CI. Both antibodies showed maximum CI saturation at 400K cells/well.

### Example 3: T2 Cell Peptide Loading and Attachment

### Materials and Methods

### Peptide Preparation and Loading

T2 cells were loaded using the relevant WT126 peptide (126-134; RMFPNAPYL) or irrelevant control WT235 peptide (235-243; CMTWNQMNL) (both from Bachem). WT126 was prepared to a stock concentration of 20 mM in sterile PBS, WT235 was prepared to a stock concentration of 20 mM in dimethyl sulfoxide (DMSO, Sigma). Cells were either loaded before or after attachment. To load before attachment, T2's were resuspended at 6×10⁶ cells/ml and the peptides added to the final desired concentration. They were loaded for 2 hours at 37° C. and 5% CO₂ in a humidified incubator. The cells were then attached to the E-plate as before using anti-CD40 at 4.5 μg/ml.

To load the cells during attachment of the cells, the peptide was added into the wells during initial 2-3 hour attachment phase.

### Results

Cells were pre-loaded with 0, 25, 50, 100 or 200 μM of WT126 or WT235 peptide and allowed to adhere to the anti-CD40 coated xCELLigence plate. There was no difference in T2 attachment in either the WT126 or WT235 loaded cells or between the different loading concentrations (FIG. 9).

### Example 4: Killing Assay

### Materials and Methods

### Effector Cell Culture

Effector cells were cultured in X-Vivo 15 (Lonza) and 5% v/v human serum (SeraLabs) supplemented with IL-2 (1.2 U/μl, Peprotech). Proliferation was stimulated by addition of CD3/CD28 DynaBeads (Life Technologies). Effector cells were cultured for 4 days before initiation of the killing assay.

### Results

### Effect Cell Mediated Killing

T2 cells were pulsed using 100 μM of WT126 peptide and allowed to attach as detailed in Examples 2 and 3. Following attachment the effector cells were added at different effector:target ratios: 0:1, 1:1, 2:1 and 5:1.

FIG. 7 shows the full cell index plot over 24 hours from T2 attachment, effector cell addition and subsequent decrease of cell index due to the cytotoxic effects. There is no killing seen in the 0:1 control, whereas the decreased CI in the other ratios indicate target cell death. The killing is proportional to the number of effector cells where the 1:1 ratio shows a decrease from 2.5 to 1.75 compared to the 2:1 and 5:1 ratios where CI is reduced to 0.87 and 0.75 respectively.

### Example 5: Impedance Assay with JY Target Cells

### Materials and Methods

### Day 1

T2, JY and WT-1 transduced cells where thawed and kept in culture for four days (37° C. and 5% CO₂). T2 and JY cells were cultured in RPMI with L-Glutamine and 20% Foetal Bovine Serum. WT-1 transduced cells were cultured in X-VIVO 10 supplemented with 5% human AB serum, IL-2 (120 IU/mL) and activated with CD3/CD28 beads.

### Day 2

### Cells Remain in Culture

### Day 3

E-96 plate was coated with Human CD40/TNFRSF5 at 4° C.

### Day 4

T2 and JY were counted and seeded at 6×10⁶ cells/mL in 24 well plates and pulsed with WT-126 (relevant peptide) or WT-235 (non-relevant peptide) or a vehicle (DMSO) at a 20 μM concentration for two hours at 37° C. and 5% CO₂. In addition, T2 were pulsed with different concentrations of WT-126 peptide (10 μM, 1 μM, 100 nM, 10 nM and 1 nM). After two hours incubation, the impedance plate was washed with PBS. T2 and JY cells were resuspended at 2×10⁶ cells/mL prior seeding 200 μL/well of the impedance plate (400.000 cells/well). Cell index was monitored until a plateau is reached—normally 1.5 h-2 h. WT-1 transduced cells were de-beaded and resuspended at 4×10⁶ cell/mL. 200 μL/well were transferred to the impedance plate (800.000 cells/well). Cell index was monitored for 24 h.

### Results

As can be seen in FIG. 10, JY cells pulsed with WT-126 were killed by effector cells, to a significantly larger amount than JY cells pulsed with WT-235.

1. A method of determining the cytotoxic effect of an effector molecule or cell on a target cell presenting a heterologous peptide comprising:
(a) incubating said effector molecule or cell with said target cell, wherein said target cell is immobilised on a substrate comprising at least one pair of electrodes; and
(b) determining the impedance during and/or after step a), wherein a decrease in impedance is indicative of a cytotoxic effect.

2. The method of claim 1 wherein said target cell is an antigen presenting cell.

3. The method of claim 1 or 2 wherein said target cell is non-adherent.

4. The method of any one of claims 1 to 3 wherein said target cell is immobilised to said substrate using a capture molecule which specifically binds to said target cell.

5. The method of claim 4 wherein said capture molecule is an antibody which specifically binds to said target cell.

6. The method of claim 5 wherein said antibody is an anti-CD40 antibody.

7. The method of claim 3 wherein said target cell is a T2 cell.

8. The method of any one of claims 1 to 7, wherein said effector molecule is an antibody or a soluble TCR.

9. The method of any one of claims 1 to 7 wherein said effector cell is an immune cell, such as a T cell or NK cell.

10. The method according to claim 9 wherein said immune cell comprises one or more receptors which specifically bind to said heterologous peptide presented by said target cell.

11. The method according to claim 10 wherein said effector cell comprises at least one TCR and/or at least one CAR which specifically bind to said heterologous peptide presented by said target cell.

12. The method according to any one of claims 1 to 11 wherein said heterologous peptide is introduced into said target cell by pulsing or is expressed in said target cell from an introduced nucleic acid comprising a nucleotide sequence which encodes the peptide.

13. The method of anyone of claims 1 to 12 which comprises an additional step before step (a) of pulsing the target cells with the heterologous peptide.

14. The method of claim 13 wherein the additional pulsing step is carried out before immobilisation of the target cells to the substrate.

15. The method of any one of claims 1 to 14 wherein said heterologous peptide is a tumour associated antigen, such as WT1 or an epitope sequence therefrom.

16. The method of claim 15 wherein said heterologous peptide comprises or consists of SEQ ID NO. 1.

17. The method of any one of claims 1 to 16 wherein the amount of heterologous peptide presented by the target cell corresponds to that presented by a diseased or non-diseased cell from a subject.

18. The method of any one of claims 1 to 17 wherein said method comprises a step of measuring of determining impedance before step (a).

19. The method of claim 18 wherein said method comprises a step of comparing the impedance measurement obtained before step (a) and the impedance measurement obtained in step (b) to determine any change in impedance.

20. Use of impedance to determine the cytotoxic effect of an effector cell and/or molecule on a target cell presenting a heterologous peptide, wherein a decrease in impedance is indicative of a cytotoxic effect.

21. The use of claim 20, wherein said target cell is immobilised to a substrate comprising at least one pair of electrodes.

22. A method for increasing the cell index of non-adherent peptide pulsed target cells on a substrate comprising at least one pair of electrodes, comprising pulsing said cells with peptide prior to attachment to said substrate.

23. A kit for use in a method of any one of claim 1 to 19 or 22, wherein said kit comprises:
(a) a target cell presenting a heterologous peptide or a target cell and a heterologous peptide or a nucleic acid comprising a nucleotide sequence encoding said heterologous peptide;
(b) a capture molecule for immobilising said target cell to a substrate;
(c) instructions for use of (a) and (b) in a method of the invention; and optionally
(d) a substrate comprising at least one pair of electrodes; and/or
(e) an effector molecule and/or cell which specifically binds to the heterologous peptide presented by the target cell, or an effector cell and a nucleic acid comprising a nucleotide sequence encoding a receptor which specifically binds to the heterologous peptide presented by the target cell.

24. A method of assessing the affinity and/or avidity of an effector cell or molecule for a heterologous peptide presented by a target cell wherein said target cell is immobilised to a substrate comprising at least one pair of electrodes comprising
a) incubating said effector cell or molecule with the immobilised target cell and
b) measuring the impedance during and/or after step a) wherein a decrease in impedance is indicative of a cytotoxic effect of the effector cell or molecule and of the effector cell or molecule having an affinity and/or avidity for the heterologous peptide.
